(19) United States
(12) Patent Application Publication
(10) Pub. No.: US 2011/0136766 A1
(43) Pub. Date: Jun. 9, 2011

(54) AURORA KINASE INHIBITORS

(75) Inventors: Linghang Zhuang, Chalfont, PA (US); David A. Claremon, Maple Glen, PA (US); Suresh B. Singh, Kendall Park, NJ (US); Lingling Shen, Belmont, MA (US); Yajun Zheng, Hockessin, DE (US); John David Lawson, Fort Washington, PA (US)

(73) Assignee: Vitae Pharmaceuticals, Inc.

(21) Appl. No.: 12/920,221

(22) PCT Filed: Mar. 4, 2009
(86) PCT No.: PCT/US09/01386

§ 371 Date: Feb. 24, 2011

Related U.S. Application Data
(60) Provisional application No. 61/068,051, filed on Mar. 4, 2008.

(51) Int. Cl. A61K 31/675; C07D 403/12; A61K 31/517; A61K 31/496; C07F 9/06; A61P 35/02; A61P 35/00

(52) U.S. Cl. 514/81; 544/284; 514/266.23; 514/252.17; 544/244

(57) ABSTRACT

Disclosed herein are Aurora kinase Inhibitors represented by Structural Formula (I): Values for the variables in Structural Formula (I) are defined herein.

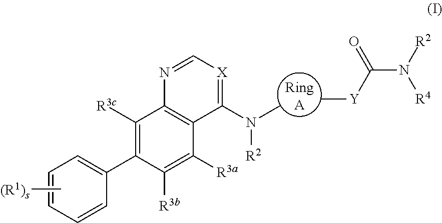

This application claims the benefit of U.S. Provisional Application Ser. No. 61/068,051, filed Mar. 4, 2008, the entire teachings of which are incorporated herein by reference.

### BACKGROUND OF THE INVENTION

The Aurora kinases are serine/threonine protein kinases, which play critical roles in the regulation of the cell cycle, especially in the late stages from the G2/M check point through the mitotic checkpoint and late mitosis. Three Aurora kinases are expressed in mammals, namely, Aurora A, B and C. Aurora-A, which localizes to centrosomes and spindle poles, has a major role in centrosome maturation and spindle assembly functioning to ensure faithful segregation of chromosomes into daughter cell. Aurora-B, a chromosome passenger protein, is associated with centromeres during prometaphase and with the spindle midzone during anaphase and telophase. It is required for histone H3 phosphorylation, correct chromosome orientation, chromosomal congression, the spindle assembly checkpoint (SAC) and cytokinesis. Aurora-C, another chromosome passenger protein, may play specific roles in male meiosis. The Aurora kinases are expressed at low level in most tissues but are highly expressed in mitotically active cells such as bone marrow, intestine, spleen, testis and thymus. Aurora A and B are only expressed and are only active as kinases during mitosis.

Aurora A and B are frequently overexpressed in many cancer cells, including breast, lung, colon, ovarian and pancreatic cells. Overexpression of Aurora A has been shown to compromise the checkpoint function that monitors spindle assembly, allowing anaphase to occur despite continued activation of the spindle checkpoint. Overexpression of Aurora B has been reported to cause endoreduplication resulting multi-nuclearity. Some evidence also suggests that overexpression of Aurora B may induce aggressive metastasis. These findings suggest that inhibition of Aurora kinases' activity may have therapeutic benefit in the treatment of cancer. Indeed, a number of small molecule Aurora kinase inhibitors, including VX-680, PHA-739358, AZD1152, MLN8054, and R763 have entered human clinical trials for suppression of tumor growth.

There is still a need for new Aurora kinase inhibitors with improved properties, such as potency.

### SUMMARY OF THE INVENTION

It has now been found that compounds represented by Structural Formula (I) are potent inhibitors of Aurora kinase A and B and cancer cell growth. For example, Compounds 1-6 (synthesized in Examples 2-7) have an IC₅₀ less than 10 nM against Aurora kinase A and less than 1.0 nM against Aurora kinase B (see Example 10). Moreover, these compounds inhibited growth of cancer cells in cell culture (see Example 11). Based on this discovery, novel Aurora kinase inhibitors, pharmaceutical compositions comprising these inhibitors and methods of treating a subject with cancer by administrating these inhibitors are disclosed herein.

One embodiment is an Aurora kinase inhibitor represented by Structural Formula (I):

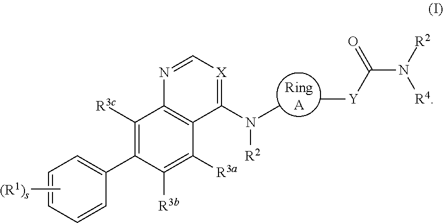

Pharmaceutically acceptable salt of Aurora kinase inhibitor represented by Structural Formula (I) are also included. The values for the variables in Structural Formulas (I) are defined in the following paragraphs:

X is CR^{3d} or N;

Y is a (C₁-C₃)alkylene or NR²;

each R¹ is independently halo, OR¹¹, S(O)ₚR¹¹, CN, NO₂, CO₂R¹¹, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹¹R¹², CONR¹¹R¹², OC(O)NR¹¹R¹², NR¹¹C(O)NR¹¹R¹², CSNR¹¹R¹², OC(S)NR¹¹R¹², NR¹¹C(S)NR¹¹R¹², SO₂NR¹¹R¹², NR¹¹ SO₂NR¹¹R¹², NR¹¹C(O)R¹², OC(O)R¹², NR¹¹C(S)R¹², NR¹¹C(O)OR¹², NR¹¹C(S)OR¹², or NR¹¹SO₂R¹²;

R^{3a} is H, halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴;

the (C₆-C₁₀)aryl and (5 to 10 membered)heteroaryl represented by R¹ and R^{3a} are optionally and independently substituted with 1 to 4 substituents selected from halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴;

the (C₃-C₈)cycloalkyl and the (3 to 9 membered)heterocyclyl represented by R¹ and R^{3a} are optionally and independently substituted with oxo, halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN;

each R² is independently H or (C₁-C₃)alkyl;

R^{3b} and R^{3d} are each independently H, halo, CN, NO₂, OH, (C₁-C₆)alkyl, halo(C₁-C₆)alkyl, (C₁-C₆)alkoxy, amino, (C₁-C₆)alkylamino, di(C₁-C₆)alkylamino, or halo(C₁-C₆)alkoxy;

R^{3c} is H or F;

R⁴ is (C₆-C₁₀)aryl, (5 to 10 membered)heteroaryl, (C₆-C₁₀)aryl(C₁-C₄)alkyl, (5 to 10 membered)heteroaryl(C₁-C₄)alkyl, (C₁-C₆)alkyl, (C₃-C₈)cycloalkyl, or (3 to 9 membered)heterocyclyl, each optionally and independently substituted with 1 to 4 substituents selected from the group consisting of halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴; or

R² and R⁴, taken together with the nitrogen to which they are attached form a 3 to 9 membered heterocycle, optionally containing 1 additional ring heteroatom selected from oxygen, nitrogen and sulfur, and optionally substituted with halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN; and

each R¹¹ and each R¹² are independently H, (C₁-C₄)alkyl, (C₃-C₈)cycloalkyl, (C₃-C₈)cycloalkyl(C₁-C₄)alkyl, halo(C₁-C₄)alkyl, (C₁-C₄)alkoxy(C₁-C₄)alkyl, or hydroxy(C₁-C₄)alkyl, wherein the (C₃-C₈)cycloalkyl and (C₃-C₈)cycloalkyl(C₁-C₄)alkyl are optionally and independently substituted with one or two groups selected from oxo, (C₁-C₂)alkyl, hydroxy, (C₁-C₂)alkoxy, amino, (C₁-C₆)alkylamino, di(C₁-C₆)alkylamino or halo; or

R¹¹ and R¹², taken together with the nitrogen atom to which they are attached, form a 3 to 9 membered nitrogen-containing heterocycle, optionally containing 1 additional ring heteroatom selected from oxygen, nitrogen and sulfur, and optionally substituted with oxo, halo, (C₁-C₄)alkyl, (C₃-C₈)cycloalkyl, spiro (C₃-C₈)cycloalkyl, (C₃-C₈)cycloalkyl(C₁-C₄)alkyl, (C₁-C₄)hydroxyalkyl, —(CH₂)_{q}—OR, (C₁-C₄)alkoxyalkyl, (C₁-C₄)alkoxy, (C₃-C₈)cycloalkoxy, (C₃-C₈)cycloalkyl(C₁-C₄)alkoxy, (C₁-C₄)thioalkyl, (C₃-C₈)cycloalkylthio, (C₃-C₈)cycloalkyl(C₁-C₄)thioalkyl, halo(C₁-C₄)alkyl, halo(C₁-C₄)alkoxy, amino, (C₁-C₄)alkylamino, (C₃-C₈)cycloalkylamino, (C₃-C₈)cycloalkyl(C₁-C₄)alkylamino, [(C₃-C₈)cycloalkyl(C₁-C₄)alkyl][(C₁-C₄)alkyl]amino, [(C₃-C₈)cycloalkyl(C₁-C₄)alkyl][(C₃-C₈)cycloalkyl]amino, di(C₁-C₄)alkylamino, di(C₃-C₈)cycloalkylamino, di[(C₃-C₈)cycloalkyl(C₁-C₄)alkyl]amino, aminocarbonyl, (C₁-C₄)alkylaminocarbonyl, (C₃-C₈)cycloalkylaminocarbonyl, (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, di(C₁-C₄)alkylaminocarbonyl, di(C₃-C₈)cycloalkylaminocarbonyl, di[(C₃-C₈)cycloalkyl(C₁-C₄)alkyl]carbonyl, (C₁-C₄)alkylcarbonyl, (C₃-C₈)cycloalkylcarbonyl, (C₃-C₈)cycloalkyl(C₁-C₄)alkylcarbonyl, (C₁-C₄)alkoxycarbonyl, (C₃-C₈)cycloalkoxycarbonyl, (C₃-C₈)cycloalkyl(C₁-C₄)alkoxycarbonyl, S(O)ₚ(C₁-C₄)alkyl, NO₂, OH, CN or 3 to 9 membered nitrogen-containing heterocyclyl optionally containing 1 additional ring heteroatom selected from oxygen, nitrogen or sulfur, and

each R¹³ and each R¹⁴ are independently H, (C₁-C₄)alkyl, halo(C₁-C₄)alkyl or hydroxy(C₁-C₄)alkyl;

s is an integer from 0 to 3;

q is an integer from 1 to 4;

p is an integer from 0 to 2;

R is P(O)(OR′)₂, P(O)(OR′)₃, S(O)(OR′), S(O)(OR′)₂, C(O)R′, C(O)N(R′)₂, P(S)(OR′)₂, P(S)(OR′)₃, S(S)(OR′), S(O)(OR′)₂, C(S)R′, and C(O)N(R′)₂;

R′ is H, (C₁-C₄)alkyl, (C₁-C₄)hydroxyalkyl, (C₁-C₄)alkoxyalkyl, or phenyl optionally substituted with halo, (C₁-C₄)alkyl, (C₁-C₄)alkoxy, amino, (C₁-C₆)alkylamino, di(C₁-C₆)alkylamino, halo(C₁-C₄)alkyl, halo(C₁-C₄)alkoxy, OH, NO₂ or CN; and

Ring A is a (C₆-C₁₀)aryl or (5-10 membered)heteroaryl, optionally substituted with halo, (C₁-C₄)alkyl, (C₁-C₄)alkoxy, amino, (C₁-C₆)alkylamino, di(C₁-C₆)alkylamino, halo(C₁-C₄)alkyl, halo(C₁-C₄)alkoxy, OH, NO₂ or CN.

Another embodiment of the invention is a pharmaceutical composition comprising an aurora kinase inhibitor represented by Structural Formula (I) or a pharmaceutically acceptable salt thereof, and a pharmaceutically acceptable carrier or diluent.

Another embodiment of the invention is a method of treating a subject with cancer comprising administering to the subject a therapeutically effective amount of an Aurora kinase inhibitor represented by Structural Formula (I) or a pharmaceutically acceptable salt thereof.

Another embodiment of the invention is an Aurora kinase inhibitor represented by Structural Formula (I) or a pharmaceutically acceptable salt thereof for use in therapy. In one embodiment, the therapy is for treating a subject with cancer.

Another embodiment of the present invention is the use of a compound represented by Structural Formula (I) or a pharmaceutically acceptable salt thereof for the manufacture of a medicament for treating a subject with cancer.

### DETAILED DESCRIPTION OF THE INVENTION

The invention is directed to Aurora kinase Inhibitors represented by Structural Formula (I) and their use in treating a subject with cancer. Values and alternative values for the variables in Structural Formula (I) are provided in the following paragraphs:

X is CR^{3d} or N. Alternatively, X is N.

Y is a (C₁-C₃)alkylene or NR². Alternatively, Y is CH₂.

Each R¹ is independently halo, OR¹¹, S(O)ₚR¹¹, CN, NO₂, CO₂R¹¹, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹¹R¹², CONR¹¹R¹², OC(O)NR¹¹R¹², NR¹¹C(O)NR¹¹R¹², CSNR¹¹R¹², OC(S)NR¹¹R¹², NR¹¹C(S)NR¹¹R¹², SO₂NR¹¹R¹², NR¹¹SO₂NR¹¹R¹², NR¹¹C(O)R¹², OC(O)R¹², NR¹¹C(S)R¹², NR¹¹C(O)OR¹², NR¹¹C(S)OR¹², or NR¹¹SO₂R¹². The (C₆-C₁₀)aryl and (5 to 10 membered)heteroaryl represented by R¹ is optionally and independently substituted with 1 to 4 substituents selected from halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴; and the (C₃-C₈)cycloalkyl and the (3 to 9 membered)heterocyclyl represented by R¹ is optionally and independently substituted with oxo, halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN.

Alternatively, each R¹ is independently halo, OR¹¹, S(O)ₚR¹¹, CN, NO₂, CO₂R¹¹, CHO, (C₁-C₆)alkyl, halo(C₁-C₄)alkyl, (C₃-C₈)cycloalkyl, (3 to 9 membered)heterocyclyl, (C₆-C₁₀)aryl, (5 to 10 membered)heteroaryl, CONR¹¹R¹², OC(O)NR¹¹R¹², NR¹¹C(O)NR¹¹R¹², SO₂NR¹¹R¹², NR¹¹SO₂NR¹¹R¹², NR¹¹C(O)OR¹², NR¹¹C(S)OR¹², or NR¹¹SO₂R¹². The (C₃-C₈)cycloalkyl and the (3 to 9 membered)heterocyclyl represented by R¹ is optionally and independently substituted with oxo, halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN. In another alternative, each R¹ is independently halo, (C₁-C₄)alkyl, (C₁-C₄)alkoxy, halo(C₁-C₄)alkyl, halo(C₁-C₄)alkoxy, CONR¹¹R¹², SO₂NR¹¹R¹², amino, (C₁-C₆)alkylamino, di(C₁-C₆)alkylamino OH, NO₂ or CN.

Each R² is independently H or (C₁-C₃)alkyl. Alternatively, R² is H.

R^{3a} is H, halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴. The (C₆-C₁₀)aryl and (5 to 10 membered)heteroaryl represented by R^{3a} is optimally and independently substituted with 1 to 4 substituents selected from halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴; and the (C₃-C₈)cycloalkyl and the (3 to 9 membered)heterocyclyl represented by R^{3a} is optionally and independently substituted with oxo, halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN.

Alternatively, R^{3a} is H, halo, OR¹³, S(O)ₚR¹³, CN, NO₂, CO₂R¹³, CHO, (C₁-C₆)alkyl, halo(C₁-C₄)alkyl, (C₃-C₈)cycloalkyl, CONR¹³R¹⁴, OC(O)NR¹³R¹⁴, NR¹³C(O)NR¹³R¹⁴, SO₂NR¹³R¹⁴, NR¹³SO₂NR¹³R¹⁴, NR¹³C(O)OR¹⁴, NR¹³C(S)OR¹⁴, or NR¹³SO₂R¹⁴. The (C₃-C₈)cycloalkyl and the (3 to 9 membered)heterocyclyl represented by R^{3a} is optionally and independently substituted with oxo, halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN. Alternatively, R^{3a} is H, halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino OH, NO₂ or CN.

R^{3b} is H, halo, CN, NO₂, OH, (C₁-C₆)alkyl, halo(C₁-C₆)alkyl, (C₁-C₆)alkoxy, amino, (C₁-C₆)alkylamino, di(C₁-C₆)alkylamino, or halo(C₁-C₆)alkoxy. Alternatively, R^{3b} is H.

R^{3c} is H or F. Alternatively, R^{3c} is H.

Each R^{3d} is independently H, halo, CN, NO₂, OH, (C₁-C₆)alkyl, halo(C₁-C₆)alkyl, (C₁-C₆)alkoxy, amino, (C₁-C₆)alkylamino, di(C₁-C₆)alkylamino, or halo(C₁-C₆)alkoxy. Alternatively, R^{3b} is H.

R⁴ is (C₆-C₁₀)aryl, (5 to 10 membered)heteroaryl, (C₆-C₁₀)aryl(C₁-C₄)alkyl, (5 to 10 membered)heteroaryl(C₁-C₄)alkyl, (C₁-C₆)alkyl, (C₃-C₈)cycloalkyl, or (3 to 9 membered)heterocyclyl, each optionally and independently substituted with 1 to 4 substituents selected from the group consisting of halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(0)R¹⁴, OC(O)R¹⁴, NR¹³C(S)R¹⁴, NR¹³C(O)OR¹⁴, NR¹³C(S)OR¹⁴, or NR¹³SO₂R¹⁴; or R² and R⁴, taken together with the nitrogen to which they are attached form a 3 to 9 membered heterocycle, optionally containing 1 additional ring heteroatom selected from oxygen, nitrogen and sulfur, and optionally substituted with halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN.

Alternatively, R⁴ is (C₆-C₁₀)aryl, (5 to 10 membered)heteroaryl, (C₆-C₁₀)aryl(C₁-C₄)alkyl, (5 to 10 membered)heteroaryl(C₁-C₄)alkyl, (C₁-C₆)alkyl, (C₃-C₈)cycloalkyl, or (3 to 9 membered)heterocyclyl, each optionally and independently substituted with 1 to 4 substituents selected from the group consisting of halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (5 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, NR¹³C(S)R¹⁴, NR¹³C(O)OR¹⁴, NR¹³C(S)OR¹⁴, or NR¹³SO₂R¹⁴. In another alternative, the group represented by R⁴ is optionally substituted with 1 to 4 substituents selected from halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino, OH, NO₂ and CN.

In yet another alternatively, R⁴ is a phenyl ring referred to as “Ring B”, wherein Ring B is optionally substituted with one to three substituents. Suitable substituents for Ring B include those described in the previous two paragraph for the aryl group represented by R⁴.

Each R¹¹ and each R¹² are independently H, (C₁-C₄)alkyl, (C₃-C₈)cycloalkyl, (C₃-C₈)cycloalkyl(C₁-C₄)alkyl, halo(C₁-C₄)alkyl, (C₁-C₄)alkoxy(C₁-C₄)alkyl, or hydroxy(C₁-C₄)alkyl, wherein the (C₃-C₈)cycloalkyl and (C₃-C₈)cycloalkyl(C₁-C₄)alkyl are optionally and independently substituted with one or two groups selected from oxo, (C₁-C₂)alkyl, hydroxy, (C₁-C₂)alkoxy, amino, (C₁-C₆)alkylamino, di(C₁-C₆)alkylamino or halo; or R¹¹ and R¹², taken together with the nitrogen atom to which they are attached, form a 3 to 9 membered nitrogen-containing heterocycle (e.g., aziridinyl, azetidinyl, pyrrolidinyl, piperazinyl, piperidinyl, azepinyl, azocinyl, azoninyl and morpholinyl, preferably aziridinyl, azetidinyl, pyrrolidinyl, piperazinyl, piperidinyl, and morpholinyl), optionally containing 1 additional ring heteroatom selected from oxygen, nitrogen and sulfur, and optionally substituted with oxo, halo, (C₁-C₄)alkyl, (C₃-C₈)cycloalkyl, spiro (C₃-C₈)cycloalkyl, (C₃-C₈)cycloalkyl(C₁-C₄)alkyl, (C₁-C₄)hydroxyalkyl, —(CH₂)_{q}—OR, (C₁-C₄)alkoxyalkyl, (C₁-C₄)alkoxy, (C₃-C₈)cycloalkoxy, (C₃-C₈)cycloalkyl(C₁-C₄)alkoxy, (C₁-C₄)thioalkyl, (C₃-C₈)cycloalkylthio, (C₃-C₈)cycloalkyl(C₁-C₄)thioalkyl, halo(C₁-C₄)alkyl, halo(C₁-C₄)alkoxy, amino, (C₁-C₄)alkylamino, (C₃-C₈)cycloalkylamino, (C₃-C₈)cycloalkyl(C₁-C₄)alkylamino, [(C₃-C₈)cycloalkyl(C₁-C₄)alkyl][(C₁-C₄)alkyl]amino, [(C₃-C₈)cycloalkyl(C₁-C₄)alkyl][(C₃-C₈)cycloalkyl]amino, di(C₁-C₄)alkylamino, di(C₃-C₈)cycloalkylamino, di[(C₃-C₈)cycloalkyl(C₁-C₄)alkyl]amino, aminocarbonyl, (C₁-C₄)alkylaminocarbonyl, (C₃-C₈)cycloalkylaminocarbonyl, (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl, di[(C₃-C₈)cycloalkyl(C₁-C₄)alkyl]carbonyl, (C₁-C₄)alkylcarbonyl, (C₃-C₈)cycloalkylcarbonyl, (C₃-C₈)cycloalkyl(C₁-C₄)alkylcarbonyl, (C₁-C₄)alkoxycarbonyl, (C₃-C₈)cycloalkoxycarbonyl, (C₃-C₈)cycloalkyl(C₁-C₄)alkoxycarbonyl, S(O)ₚ(C₁-C₄)alkyl, NO₂, OH, CN or 3 to 9 membered nitrogen-containing heterocyclyl (e.g., aziridinyl, azetidinyl, pyrrolidinyl, piperazinyl, piperidinyl, azepinyl, azocinyl, azoninyl and morpholinyl, preferably aziridinyl, azetidinyl, pyrrolidinyl, piperazinyl, piperidinyl, and morpholinyl) optionally containing 1 additional ring heteroatom selected from oxygen, nitrogen or sulfur. Alternatively, the 3 to 9 membered heterocycle formed from R¹¹ and R¹², taken together with the nitrogen atom to which they are attached is: i) optionally substituted at any substitutable ring carbon atoms with halogen, oxo, (C₁-C₄)alkyl, Spiro (C₃-C₈)cycloalkyl, (C₃-C₈)cycloalkyl, hydroxy, (C₁-C₄)alkoxy or three to seven membered nitrogen-containing heterocylyl (e.g., aziridinyl, azetidinyl, pyrrolidinyl, piperazinyl, piperidinyl, azepinyl, azocinyl, azoninyl and morpholinyl, preferably aziridinyl, azetidinyl, pyrrolidinyl, piperazinyl, piperidinyl, and morpholinyl) optionally containing one additional ring heteroatom selected from nitrogen and oxygen; and ii) optionally substituted at any substitutable ring nitrogen atom with (C₁-C₄)alkyl, (C₁-C₄)haloalkyl, (C₃-C₈)cycoalkyl, (C₃-C₈)cycoalkyl(C₁-C₄)alkyl, hydroxy(C₁-C₄)alkyl, —(CH₂)_{q}—OR, (C₁-C₄)alkoxyalkyl, (C₁-C₄)alkylcarbonyl, (C₃-C₈)cycloalkylcarbonyl, (C₃-C₈)cycloalkyl(C₁-C₄)alkylcarbonyl, (C₁-C₄)alkylaminocarbonyl, (C₃-C₈)cycloalkylaminocarbonyl (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl or di[(C₃-C₈)cycloalkyl(C₁-C₄)alkyl]carbonyl.

Alternatively, R¹¹ and R¹², taken together with the nitrogen atom to which they are attached, form a 3 to 9 membered nitrogen-containing heterocycle (e.g., aziridinyl, azetidinyl, pyrrolidinyl, piperazinyl, piperidinyl, azepinyl, azocinyl, azoninyl and morpholinyl, preferably aziridinyl, azetidinyl, pyrrolidinyl, piperazinyl, piperidinyl, and morpholinyl), optionally containing 1 additional ring heteroatom selected from oxygen, nitrogen and sulfur, and optionally substituted with oxo, halo, (C₁-C₄)alkyl, (C₃-C₈)cycloalkyl, spiro (C₃-C₈)cycloalkyl, (C₃-C₈)cycloalkyl(C₁-C₄)alkyl, (C₁-C₄)hydroxyalkyl, —(CH₂)_{q}—OR, (C₁-C₄)alkoxyalkyl, (C₁-C₄)alkoxy, (C₃-C₈)cycloalkoxy, (C₃-C₈)cycloalkyl(C₁-C₄)alkoxy, (C₁-C₄)thioalkyl, (C₃-C₈)cycloalkylthio, (C₃-C₈)cycloalkyl(C₁-C₄)thioalkyl, halo(C₁-C₄)alkyl, halo(C₁-C₄)alkoxy, amino, (C₁-C₄)alkylamino, (C₃-C₈)cycloalkylamino, (C₃-C₈)cycloalkyl(C₁-C₄)alkylamino, [(C₃-C₈)cycloalkyl(C₁-C₄)alkyl][(C₁-C₄)alkyl]amino, [(C₃-C₈)cycloalkyl(C₁-C₄)alkyl][(C₃-C₈)cycloalkyl]amino, di(C₁-C₄)alkylamino, di(C₃-C₈)cycloalkylamino, di[(C₃-C₈)cycloalkyl(C₁-C₄)alkyl]amino, aminocarbonyl, (C₁-C₄)alkylaminocarbonyl, (C₃-C₈)cycloalkylaminocarbonyl, (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl, di[(C₃-C₈)cycloalkyl(C₁-C₄)alkyl]carbonyl, (C₁-C₄)alkylcarbonyl, (C₃-C₈)cycloalkylcarbonyl, (C₃-C₈)cycloalkyl(C₁-C₄)alkylcarbonyl, (C₁-C₄)alkoxycarbonyl, (C₃-C₈)cycloalkoxycarbonyl, (C₃-C₈)cycloalkyl(C₁-C₄)alkoxycarbonyl, S(O)ₚ(C₁-C₄)alkyl, NO₂, OH, CN or 3 to 9 membered nitrogen-containing heterocyclyl (e.g., aziridinyl, azetidinyl, pyrrolidinyl, piperazinyl, piperidinyl, azepinyl, azocinyl, azoninyl and morpholinyl, preferably aziridinyl, azetidinyl, pyrrolidinyl, piperazinyl, piperidinyl, and morpholinyl) optionally containing 1 additional heteroatom selected from oxygen, nitrogen or sulfur. Alternatively, the 3 to 9 membered heterocycle (e.g., aziridinyl, azetidinyl, pyrrolidinyl, piperazinyl, piperidinyl, azepinyl, azocinyl, azoninyl and morpholinyl, preferably aziridinyl, azetidinyl, pyrrolidinyl, piperazinyl, piperidinyl, and morpholinyl) formed from R¹¹ and R¹², taken together with the nitrogen atom to which they are attached is: i) optionally substituted at any substitutable ring carbon atoms with halogen, oxo, (C₁-C₄)alkyl, spiro (C₃-C₈)cycloalkyl, (C₃-C₈)cycloalkyl, hydroxy, (C₁-C₄)alkoxy or three to seven membered nitrogen-containing heterocylyl (e.g., aziridinyl, azetidinyl, pyrrolidinyl, piperazinyl, piperidinyl, azepinyl, azocinyl, azoninyl and morpholinyl, preferably aziridinyl, azetidinyl, pyrrolidinyl, piperazinyl, piperidinyl, and morpholinyl)optionally containing one additional ring heteroatom selected from nitrogen and oxygen; and ii) optionally substituted at any substitutable ring nitrogen atom with (C₁-C₄)alkyl, (C₁-C₄)haloalkyl, (C₃-C₈)cycoalkyl, (C₃-C₈)cycoalkyl(C₁-C₄)alkyl, hydroxy(C₁-C₄)alkyl, —(CH₂)_{q}—OR, (C₁-C₄)alkoxyalkyl, (C₁-C₄)alkylcarbonyl, (C₃-C₈)cycloalkylcarbonyl, (C₃-C₈)cycloalkyl(c₁-C₄)alkylcarbonyl, (C₁-C₄)alkylaminocarbonyl, (C₃-C₈)cycloalkylaminocarbonyl (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl or di[(C₃-C₈)cycloalkyl(C₁-C₄)alkyl]carbonyl.

Each R¹³ and each R¹⁴ are independently H, (C₁-C₄)alkyl, halo(C₁-C₄)alkyl or hydroxy(C₁-C₄)alkyl. Alternatively, each R¹³ and each R¹⁴ are independently H or (C₁-C₄)alkyl.

s is an integer from 0 to 3. Alternatively, s is 1 or 2.

q is an integer from 1 to 4.

p is an integer from 0 to 2.

R is P(O)(OR′)₂, P(O)(OR′)₃, S(O)(OR′), S(O)(OR′)₂, C(O)R′, C(O)N(R′)₂, P(S)(OR′)₂, P(S)(OR′)₃, C(S)R′, or C(O)N(R′)₂.

R′ is H, (C₁-C₄)alkyl, (C₁-C₄)hydroxyalkyl, (C₁-C₄)alkoxyalkyl, or phenyl optionally substituted with halo, (C₁-C₄)alkyl, (C₁-C₄)alkoxy, amino, (C₁-C₆)alkylamino, di(C₁-C₆)alkylamino, halo(C₁-C₄)alkyl, halo(C₁-C₄)alkoxy, OH, NO₂ or CN. Alternatively, R′ is H or (C₁-C₄)alkyl.

Ring A is a (C₆-C₁₀)aryl or (5-10 membered)heteroaryl, optionally substituted with halo, (C₁-C₄)alkyl, (C₁-C₄)alkoxy, amino, (C₁-C₆)alkylamino, di(C₁-C₆)alkylamino, halo(C₁-C₄)alkyl, halo(C₁-C₄)alkoxy, OH, NO₂ or CN. Alternatively, Ring A is a pyrazolyl represented by

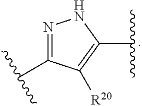

R²⁰ is H, halo, (C₁-C₄)alkyl, (C₁-C₄)alkoxy, halo(C₁-C₄)alkyl, halo(C₁-C₄)alkoxy, NH₂, (C₁-C₄)alkylamine, di(C₁-C₄)alkylamine, OH, NO₂ or CN. Alternatively, R²⁰ is H or (C₁-C₄)alkyl. In another alternative, R²⁰ is H.

In a second embodiment the Aurora kinase inhibitor of the invention is represented by a structural formula selected from any one of Structural Formulas (II)-XIII):

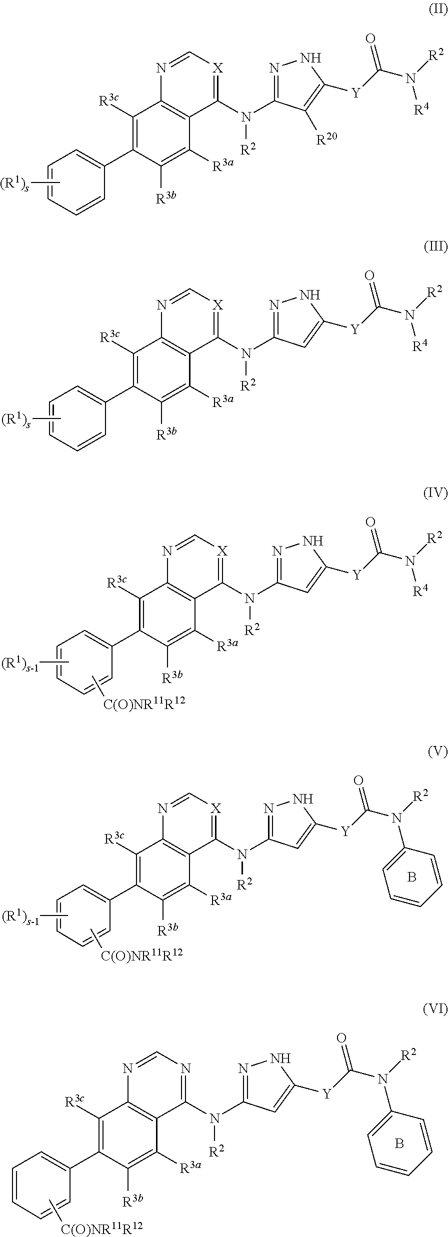

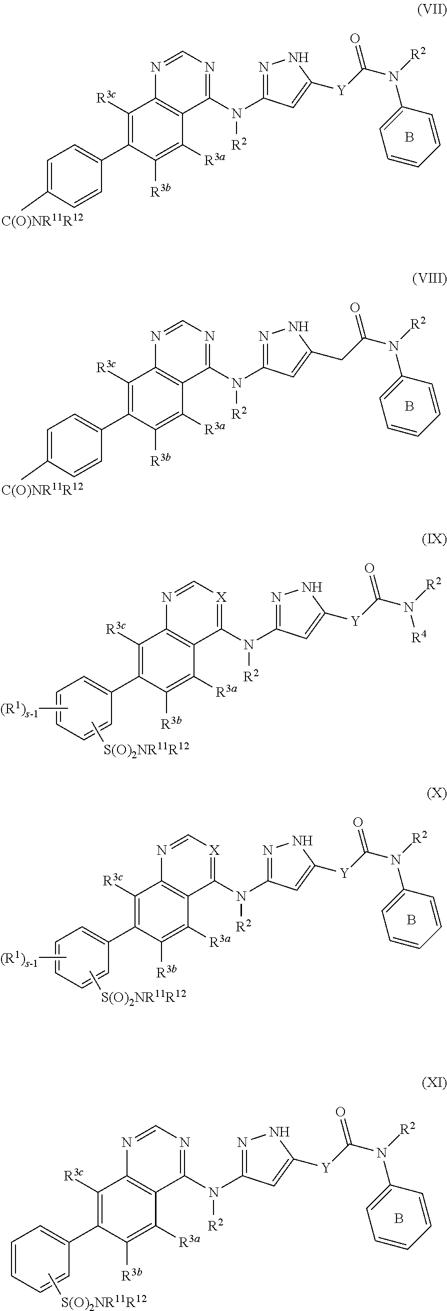

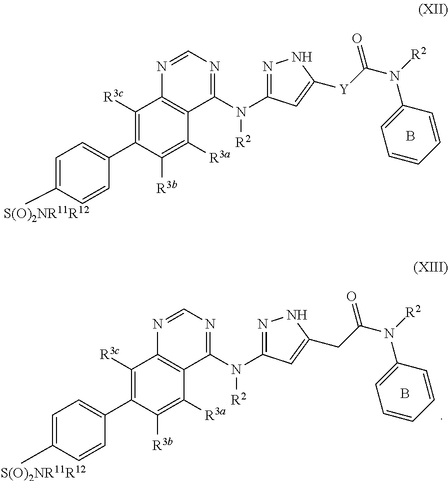

Pharmaceutically acceptable salts of the Aurora kinase inhibitors disclosed herein (including those represented by any one of Structural Formulas (II)-(XIII)) are also included in the invention. Values for the variables in these structural formulas are provided in the following paragraphs:

R²⁰ in Structural Formula (II) is H, halo, (C₁-C₄)alkyl, (C₁-C₄)alkoxy, halo(C₁-C₄)alkyl, halo(C₁-C₄)alkoxy, NH₂, (C₁-C₄)alkylamine, di(C₁-C₄)alkylamine, OH, NO₂ or CN;

Ring B in Structural Formulas (V)-(VIII) and (X)-(XIII) is optionally substituted with one to three substituents. Exemplary substituents for Ring B are as described for the aryl and heteroaryl group represented by R⁴. Alternatively, substituents for Ring B include halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino, OH, NO₂ and CN; and

values and alternative values for the remainder of the variables in Structural Formulas (II)-(XIII) are as described above for Structural Formula (I).

In a third embodiment the Aurora kinase inhibitor of the invention is represented by a structural formula selected from any one of Structural Formulas (I)-XIII), wherein the values for each of the variables in the structural formulas are defined below:

the 3 to 9 membered heterocycle formed from R¹¹ and R¹², taken together with the nitrogen atom to which they are attached is: i) optionally substituted at any substitutable ring carbon atoms with halogen, oxo, (C₁-C₄)alkyl, spiro (C₃-C₈)cycloalkyl, (C₃-C₈)cycloalkyl, hydroxy, (C₁-C₄)alkoxy or three to seven membered nitrogen-containing heterocylyl optionally containing one additional ringheteroatom selected from nitrogen and oxygen; and ii) optionally substituted at any substitutable ring nitrogen atom with (C₁-C₄)alkyl, (C₁-C₄)haloalkyl, (C₃-C₈)cycoalkyl, (C₃-C₈)cycoalkyl(C₁-C₄)alkyl, hydroxy(C₁-C₄)alkyl, —(CH₂)_{q}—OR, (C₁-C₄)alkoxyalkyl, (C₁-C₄)alkylcarbonyl, (C₃-C₈)cycloalkylcarbonyl, (C₃-C₈)cycloalkyl(c₁-C₄)alkylcarbonyl, (C₁-C₄)alkylaminocarbonyl, (C₃-C₈)cycloalkylaminocarbonyl (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl or di[(C₃-C₈)cycloalkyl(C₁-C₄)alkyl]carbonyl;

R²⁰ in Structural Formula (II) is H, halo, (C₁-C₄)alkyl, (C₁-C₄)alkoxy, halo(C₁-C₄)alkyl, halo(C₁-C₄)alkoxy, NH₂, (C₁-C₄)alkylamine, di(C₁-C₄)alkylamine, OH, NO₂ or CN;

Ring B in Structural Formulas (V)-(VIII) and (X)-(XIII) is optionally substituted with one to three substituents. Exemplary substituents for Ring B are as described for the aryl and heteroaryl group represented by R⁴. Alternatively, substituents for Ring B include halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino, OH, NO₂ and CN; and

values and alternative values for the remainder of the variables in Structural Formulas (II)-(XIII) are as described above for Structural Formula (I).

In a fourth embodiment the Aurora kinase inhibitor of the invention is represented by a structural formula selected from any one of Structural Formulas (I)-XIII), wherein the values for each of the variables in the structural formulas are defined below:

each R¹ is independently halo, OR¹¹, S(O)ₚR¹¹, CN, NO₂, CO₂R¹¹, CHO, (C₁-C₆)alkyl, halo(C₁-C₄)alkyl, (C₃-C₈)cycloalkyl, (3 to 9 membered)heterocyclyl, (C₆-C₁₀)aryl, (5 to 10 membered)heteroaryl, CONR¹¹R¹², OC(O)NR¹¹R¹², NR¹¹C(O)NR¹¹R¹², SO₂NR¹¹R¹², NR¹¹SO₂NR¹¹R¹², NR¹¹C(O)OR¹², NR¹¹C(S)OR¹², or NR¹¹SO₂R¹²;

R^{3a} is H, halo, OR¹³, S(O)ₚR¹³, CN, NO₂, CO₂R¹³, CHO, (C₁-C₆)alkyl, halo(C₁-C₄)alkyl, (C₃-C₈)cycloalkyl, CONR¹³R¹⁴, OC(O)NR¹³R¹⁴, NR¹³C(O)NR¹³R¹⁴, SO₂NR¹³R¹⁴, NR¹³SO₂NR¹³R¹⁴, NR¹³C(O)OR¹⁴, NR¹³C(S)OR¹⁴, or NR¹³SO₂R¹⁴;

the (C₃-C₈)cycloalkyl and the (3 to 9 membered)heterocyclyl represented by R¹ and R^{3a} are optionally and independently substituted with oxo, halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN;

the 3 to 9 membered heterocycle formed from R¹¹ and R¹², taken together with the nitrogen atom to which they are attached is: i) optionally substituted at any substitutable ring carbon atoms with halogen, oxo, (C₁-C₄)alkyl, spiro (C₃-C₈)cycloalkyl, (C₃-C₈)cycloalkyl, hydroxy, (C₁-C₄)alkoxy or three to seven membered nitrogen-containing heterocylyl optionally containing one additional ringheteroatom selected from nitrogen and oxygen; and ii) optionally substituted at any substitutable ring nitrogen atom with (C₁-C₄)alkyl, (C₁-C₄)haloalkyl, (C₃-C₈)cycoalkyl, (C₃-C₈)cycoalkyl(C₁-C₄)alkyl, hydroxy(C₁-C₄)alkyl, —(CH₂)_{q}—OR, (C₁-C₄)alkoxyalkyl, (C₁-C₄)alkylcarbonyl, (C₃-C₈)cycloalkylcarbonyl, (C₃-C₈)cycloalkyl(c₁-C₄)alkylcarbonyl, (C₁-C₄)alkylaminocarbonyl, (C₃-C₈)cycloalkylaminocarbonyl (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl or di[(C₃-C₈)cycloalkyl(C₁-C₄)alkyl]carbonyl;

R²⁰ in Structural Formula (II) is H, halo, (C₁-C₄)alkyl, (C₁-C₄)alkoxy, halo(C₁-C₄)alkyl, halo(C₁-C₄)alkoxy, NH₂, (C₁-C₄)alkylamine, di(C₁-C₄)alkylamine, OH, NO₂ or CN;

Ring B in Structural Formulas (V)-(VIII) and (X)-(XIII) is optionally substituted with one to free substituents. Exemplary substituents for Ring B are as described for the aryl and heteroaryl group represented by R⁴. Alternatively, substituents for Ring B include halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino, OH, NO₂ and CN; and

values and alternative values for the remainder of the variables in Structural Formulas (II)-(XIII) are as described above for Structural Formula (I). Alternatively, each R¹ is independently halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino, CONR¹¹R¹², SO₂NR¹¹R¹², OH, NO₂ or CN; R^{3a} is H, halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino OH, NO₂ or CN; and R^{3b} and R^{3c} are each H; and values and alternative values for the remainder of the variables are as just described.

In a fifth embodiment the Aurora kinase inhibitor of the invention is represented by a structural formula selected from any one of Structural Formulas (I)-(III) or (IX), wherein the values for each of the variables in the structural formulas are defined below:

R⁴ is (C₆-C₁₀)aryl, (5 to 10 membered)heteroaryl, (C₆-C₁₀)aryl(C₁-C₄)alkyl, (5 to 10 membered)heteroaryl(C₁-C₄)alkyl, (C₁-C₆)alkyl, (C₃-C₈)cycloalkyl, or (3 to 9 membered)heterocyclyl, each optionally and independently substituted with 1 to 4 substituents selected from the group consisting of halo, OR¹¹, S(O)ₚR¹¹, CN, NO₂, CO₂R¹¹, CHO, (C₁-C₆)alkyl, halo(C₁-C₆)alkyl, (C₃-C₈)cycloalkyl, (5 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, NR¹³C(S)R¹⁴, NR¹³C(O)OR¹⁴, NR¹³C(S)OR¹⁴, or NR¹³SO₂R¹⁴; and values and alternative values for the remainder of the variables are as described above for Structural Formula (I), of in the first, second, third or fourth embodiment. Alternatively, R⁴ is phenyl optionally substituted with 1 to 4 substituents selected from the group consisting of halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (5 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, NR¹³C(S)R¹⁴, NR¹³C(O)OR¹⁴, NR¹³C(S)OR¹⁴, or NR¹³SO₂R¹⁴. In another alternative, R⁴ is phenyl optionally substituted with 1 to 4 substituents selected from the group consisting of halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino, OH, NO₂ and CN.

Specific Aurora kinase inhibitors are provided in Examples 2-8 and 10-16 (Compound 1-7 and 9-15). Other specific Aurora kinase inhibitors are shown below:

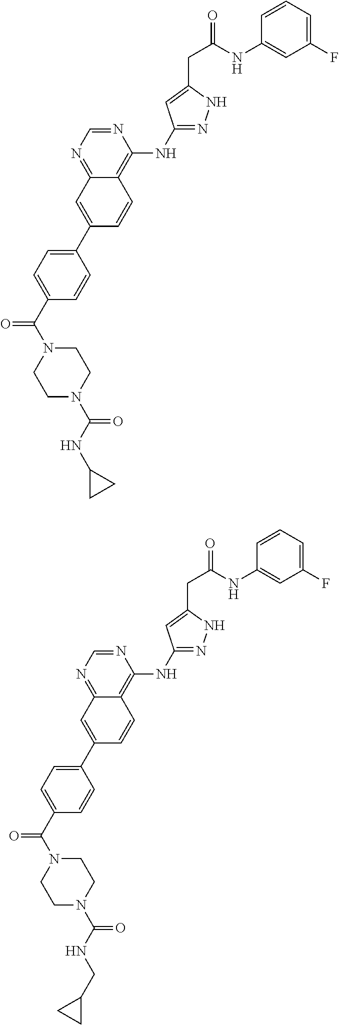

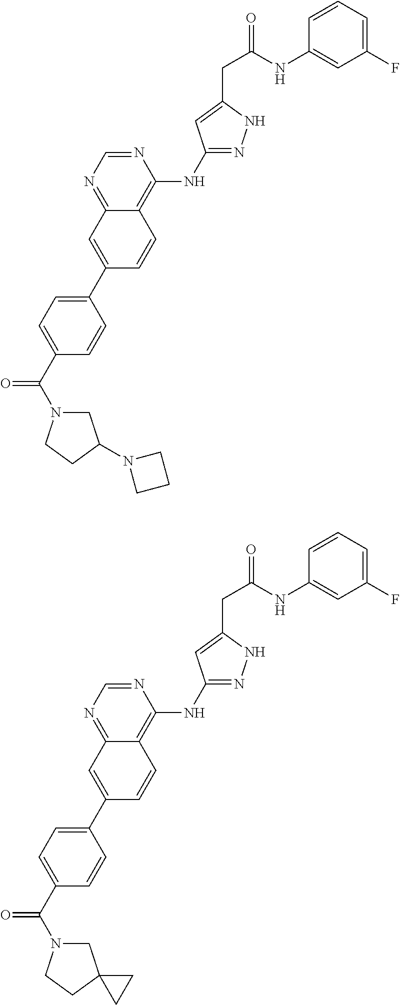

Pharmaceutically acceptable salts of the above Aurora kinase inhibitors are also included in the invention.

When any variable (e.g., aryl, heterocyclyl, R¹¹, R¹², R¹³, R¹⁴, etc.) occurs more than once in a compound, its definition on each occurrence is independent of any other occurrence.

“Alkyl”, used alone or as part of a larger moiety such as “alkoxy”, “hydroxyalkyl”, “alkoxyalkyl”, “alkylamine”, “dialkyamine”, “alkoxycarbonyl” or “alkylaminocarbonyl” means a saturated branched or straight-chain monovalent hydrocarbon having the specified number of carbon atoms. Thus, “(C₁-C₈)alkyl” means a saturated hydrocarbon having from 1-8 carbon atoms in a linear or branched arrangement. “(C₁-C₆)alkyl” includes methyl, ethyl, propyl, butyl, pentyl, and hexyl. If the number of carbon atoms in an alkyl group is not specified, “alkyl” means from one to eight carbon atoms.

“Alkylene” means —[CH₂]ₓ—, wherein x is a positive integer. x is typically a positive integer from 1-3.

“Cycloalkyl”, used alone or as part of a larger moiety such as “cycloalkylalkyl” or “cycloalkoxyalkyl” means a saturated cyclic hydrocarbon having the specified number of carbon atoms. Thus, (C₃-C₈)cycloalkyl means a having from 3-8 carbon atoms arranged in a ring. (C₃-C₈)cycloalkyl includes cyclopropyl, cyclobutyl, cyclopentyl, cyclohexyl, and cycloheptyl. “Cycloalkylalkyl” is an alkyl group substituted with a cycloalkyl group. When the number of carbon atoms in a cycloalkyl is not specified, “cycloalkyl” has from three to eight ring carbon atoms.

A “spiro” cycloalkyl group is a cycloalkyl group which shares one ring carbon with another alkyl or cycloalkyl group. When not designated as “spiro”, a cycloalkyl group shares no ring atom with the group to which it is bonded.

Haloalkyl and halocycloalkyl include mono, poly, and perhaloalkyl groups where the halogens are independently selected from fluorine, chlorine, bromine and iodine.

“Heterocyclyl”, used alone or as part of a larger moiety such as “heterocyclyalkyl”, is a non-aromatic 3-9-membered (preferably 3-7 membered, such as aziridinyl, azetidinyl, pyrrolidinyl, piperazinyl, piperidinyl, azepinyl and morpholinyl) non-aromatic heterocyclic rings (saturated or partially unsaturated) containing 1 to 4 ring heteroatoms independently selected from N, O, and S, and include aziridinyl, azetidinyl, pyrrolidinyl, piperazinyl, piperidinyl, azepinyl, azocinyl, azoninyl, tetrahydrofuranyl, tetrahydropyranyl, tetrahydrothienyl, tetrahydrothiopyranyl, isoxazolidinyl, 1,3-dioxolanyl, 1,3-dithiolanyl, 1,3-dioxanyl, 1,4-dioxanyl, 1,3-dithianyl, 1,4-dithianyl, morpholinyl, thiomorpholinyl, thiomorpholinyl 1,1-dioxide, tetrahydro-2H-1,2-thiazinyl 1,1-dioxide, and isothiazolidinyl 1,1-dioxide. Also included are oxo substituted saturated heterocyclic rings such as tetrahydrothienyl 1-oxide, tetrahydrothiophene 1,1-dioxide, thiomorpholine 1-oxide, thiomorpholine 1,1-dioxide, tetrahydro-2H-1,2-thiazinyl 1,1-dioxide, and isothiazolidinyl 1,1-dioxide, pyrrolidinyl-2-one, piperidinyl-2-one, piperazinyl-2-one, and morpholinyl-2-one. The terms “heterocyclyl”, “heterocycle” and “heterocyclic ring” are used interchangeably herein. “Heterocyclyalkyl” means alky substituted with heterocycly.

“Heteroaryl”, used alone or as part of a larger moiety as in “heteroaralkyl” or “heteroarylalkoxy”, means a 5-10 membered monovalent heteroaromatic monocyclic and polycylic ring radical containing 1 to 4 heteroatoms independently selected from N, O, and S. The term “heteroaryl” also includes monocyclic heteroaryl ring fused to non-aromatic carbocyclic ring or to a heterocyclyl group. Heteroaryl groups include furyl, thienyl, thiophenyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridinyl-N-oxide, pyridazinyl, pyrimidinyl, pyrazinyl, indolizinyl, indolyl, isoindolyl, benzo[b]furyl, benzo[b]thienyl, indazolyl, benzimidazolyl, benzthiazolyl, purinyl, 4H-quinolizinyl, quinolinyl, isoquinolinyl, quinazolinyl, benzothienyl, benzofuranyl, 2,3-dihydrobenzofuranyl, benzodioxolyl, benzimidazolyl, indazolyl, benzisoxazolyl, benzoxazolyl, benzothiazolyl, cinnolinyl, phthalzinyl, quinazolinyl, quinoxalinyl, 1,8-naphthyridinyl, 1,2,3-triazolyl, 1,2,4-triazolyl, 1,3,4-oxadiazolyl, 1,2,5-thiadiazolyl, 1,2,5-thiadiazolyl-1-oxide, 1,2,5-thiadiazolyl-1,1-dioxide, 1,3,4-thiadiazolyl, 1,2,4-triazinyl, 1,3,5-triazinyl, tetrazolyl, and pteridinyl. The terms “heteroaryl”, “heteroaromatic”, “heteroaryl ring”, “heteroaryl group” and “heteroaromatic group” are used interchangeably herein. “Heteroaralkyl” means alkyl substituted with heteroaryl; and “heteroarylalkoxy” —OR wherein R is heteroaryl.

“Alkoxy” means —OR where R is an alkyl group. Examples include the methoxy, ethoxy, propoxy, and butoxy. “Cycloalkoxy” means —OR where R is a cycloalkyl group. “Cycloalkylalkyloxy” means —OR wherein R is cycloalkylalkyl group.

“Amino” means —NH₂; “alkylamine” and “dialkylamine” mean —NHR and —NR₂, respectively, wherein R is an alkyl group. “Cycloalkylamine” and “dicycloalkylamine” mean —NHR and —NR₂, respectively, wherein R is a cycloalkyl group. “Cycloalkylalkylamine” means —NHR wherein R is a cycloalkylalkyl group. “[Cycloalkylalkyl][alkyl]amine” means —N(R)₂ wherein one R is cycloalkylalkyl and the other R is alkyl.

“Thioalkyl” means —SR wherein R is alkyl. “Thiocycloalkyl” means —SR wherein R is cycloalkyl.

“Aminocarbonyl” means —C(O)NH₂. “Alkylaminocarbonyl” and “dialkylaminocarbonyl” mean —C(O)NHR and —C(O)NR₂, respectively, wherein R is alkyl. “Cycloalkylaminocarbonyl” and “dicycloalkylaminocarbonyl” mean —C(O)NHR and —C(O)NR₂, respectively, wherein R is cycloalkyl. “Cycloalkylalkylaminocarbonyl” means —C(O)NHR, wherein R is cycloalkylalkyl. “[Cycloalkylalkyl][alkyl] aminocarbonyl: means —C(O)N(R)₂, wherein one R is cycloalkylalkyl and the other is alkyl.

“Alkylcarbonyl” is —C(O)R where R is alkyl. “Cycloalkylcarbonyl” is —C(O)R where R is cycloalkyl.

“Hydroxyalkyl” and “alkoxyalkyl” are alkyl groups substituted with hydroxy and alkoxy, respectively.

“Alkoxycarbonyl” is —C(O)OR wherein R is alkyl. “Cycloalkoxycarbonyl” is —C(O)OR wherein R is cycloalkyl.

“Aryl”, used alone or as part of a larger moiety as in “aralkyl”, “aralkoxy”, or “aryloxyalkyl”, means a 6-10 membered carbocyclic aromatic monocyclic or polycyclic ring system. Examples include phenyl and naphthalenyl. The term “aryl” also includes phenyl rings fused to non-aromatic carbocyclic ring or to a heterocyclyl group. The term “aryl” may be used interchangeably with the terms “aromatic group”, “aryl ring” “aromatic ring”, “aryl group” and “aromatic group”.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Oxo” refers to ═O. When an oxo group is a substituent on a carbon atom, they form a carbonyl group (—C(O)—). When one oxo group is a substituent on a sulfur atom, they form a sulfinyl (sulfoxide —S(O)—) group. When two oxo groups are a substituent on a sulfur atom, they form a sulfonyl (sulfone —S(O)₂—) group.

The term “ring atom” is an atom such as C, N, O or S that is in the ring of an aryl group, heteroaryl group, cycloalkyl group or heterocyclyl group. A “substitutable ring atom” in an aryl, heteroaryl or heterocyclyl is a carbon or nitrogen atom in the aryl, heteroaryl or heterocyclyl that is bonded to at least one hydrogen atom. The hydrogen(s) can be optionally replaced with a suitable substituent group. Thus, the term “substitutable ring atom” does not include ring carbon or nitrogen atoms when the structure depicts that they are not attached to any hydrogen atoms. For example, the carbon atom attached to the —C(O)NR¹¹R¹² group in Structural Formula (VIII) is not a substitutable ring carbon atom.

Suitable substituents for an alkyl, aryl, heteroaryl and heterocyclyl are those which do not significantly reduce the ability of the compound to inhibit the activity of Aurora kinase(s). Unless otherwise specified, suitable substituents for an alkyl, aryl, heteroaryl and heterocyclyl include halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴, wherein R¹³ and R¹⁴ are independently H, (C₁-C₄)alkyl, halo(C₁-C₄)alkyl or hydroxy(C₁-C₄)alkyl. Unless otherwise specified, preferred substituents for alkyl, aryl, heteroaryl and heterocyclyl include halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino OH, NO₂ or CN.

The disclosed Aurora kinase inhibitors may contain one or more chiral center and/or double bond and, therefore, exist as stereoisomers, such as double-bond isomers (i.e., geometric isomers), enantiomers, and/or diastereomers. When compounds of the invention are depicted or named without indicating the stereochemistry, it is to be understood that both stereomerically pure forms (e.g., geometrically pure, enantiomerically pure, or diastereomerically pure) and stereoisomeric mixtures are encompassed.

The invention encompasses all geomerically-pure forms and geomerically-enriched (i.e. greater than 50% of either E or Z isomer) mixtures, of the disclosed Aurora kinase inhibitors. Mixtures include 1:20, 1:10, 20:80, 30:70, 40:60 and 50:50 E:Z and Z:E ratios by mole.

As used herein, a racemic mixture means 50% of one enantiomer and 50% of is corresponding enantiomer relative to all chiral centers in the molecule. The invention encompasses all enantiomerically-pure, enantiomerically-enriched, diastereomerically pure, diastereomerically enriched, and racemic mixtures, and diastereomeric mixtures of the disclosed Aurora kinase inhibitors which have chiral center(s).

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

When the stereochemistry of the disclosed Aurora kinase inhibitors are named or depicted by structure, the named or depicted stereoisomer is at least 60%, 70%, 80%, 90%, 99% or 99.9% by weight pure relative to the other stereoisomers. When a single stereoisomer is named or depicted by structure, the depicted or named stereoisomer is at least 60%, 70%, 80%, 90%, 99% or 99.9% pure. Percent purity by weight is the ratio of the weight of the named stereoisomer over the weight of the named stereoisomer plus the weight of its stereoisomers.

Pharmaceutically acceptable salts of the Aurora kinase inhibitors disclosed herein are included in the present invention. For example, an acid salt of an Aurora kinase inhibitor containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

Salts of the Aurora kinase inhibitors containing an acidic functional group can be prepared by reacting with a suitable base. Such a pharmaceutically acceptable salt may be, made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

When a disclosed Aurora kinase inhibitor is named or depicted by structure, it is to be understood that solvates (e.g., hydrates) of the Aurora kinase inhibitor or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates”. Hydrates include stoichiometric hydrates as well as compositions containing variable amounts, of water.

When a disclosed Aurora kinase inhibitor is named or depicted by structure, it is to be understood that the compound, including solvates thereof, may exist in crystalline forms, non-crystalline forms or a mixture thereof. The Aurora kinase inhibitor or solvates may also exhibit polymorphism (i.e. the capacity to occur in different crystalline forms). These different crystalline forms are typically known as “polymorphs.” It is to be understood that when named or depicted by structure, the disclosed Aurora kinase inhibitors and solvates (e.g., hydrates) also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

Cancers that can be treated or prevented by the methods of the present invention include lung cancer, breast cancer, colon cancer, brain cancer, neuroblastoma, prostate cancer, melanoma, glioblastoma multiform, ovarian cancer, lymphoma, leukemia, melanoma, sarcoma, paraneoplasia, osteosarcoma, germinoma, glioma and mesothelioma. In one specific embodiment, the cancer is lung cancer, colon cancer, brain cancer, neuroblastoma, prostate cancer, melanoma, glioblastoma mutiform or ovarian cancer. In another specific embodiment, the cancer is lung cancer, breast cancer, colon cancer, brain cancer, neuroblastoma, prostate cancer, melanoma, glioblastoma multiform or ovarian cancer. In yet another specific embodiment, the cancer is a breast cancer. In yet another specific embodiment, the cancer is a basal sub-type breast cancer or a luminal B sub-type breast cancer. In yet another specific embodiment, the cancer is a soft tissue cancer. A “soft tissue cancer” is an art-recognized term that encompasses tumors derived from any soft tissue of the body. Such soft tissue connects, supports, or surrounds various structures and organs of the body, including, but not limited to, smooth muscle, skeletal muscle, tendons, fibrous tissues, fatty tissue, blood and lymph vessels, perivascular tissue, nerves, mesenchymal cells and synovial tissues. Thus, soft tissue cancers can be of fat tissue, muscle tissue, nerve tissue, joint tissue, blood vessels, lymph vessels, and fibrous tissues. Soft tissue cancers can be benign or malignant. Generally, malignant soft tissue cancers are referred to as sarcomas, or soft tissue sarcomas. There are many types of soft tissue tumors, including lipoma, lipoblastoma, hibernoma, liposarcoma, leiomyoma, leiomyosarcoma, rhabdomyoma, rhabdomyosarcoma, neurofibroma, schwannoma (neurilemoma), neuroma, malignant schwannoma, neurofibrosarcoma, neurogenic sarcoma, nodular tenosynovitis, synovial sarcoma, hemangioma, glomus tumor, hemangiopericytoma, hemangioendothelioma, angiosarcoma, Kaposi sarcoma, lymphangioma, fibroma, elastofibroma, superficial fibromatosis, fibrous histiocytoma, fibrosarcoma, fibromatosis, dermatofibrosarcoma protuberans (DFSP), malignant fibrous histiocytoma (MFH), myxoma, granular cell tumor, malignant mesenchymomas, alveolar soft-part sarcoma, epithelioid sarcoma, clear cell sarcoma, and desmoplastic small cell tumor. In a particular embodiment, the soft tissue cancer is a sarcoma selected from the group consisting of a fibrosarcoma, a gastrointestinal sarcoma, a leiomyosarcoma, a dedifferentiated liposarcoma, a pleomorphic liposarcoma, a malignant fibrous histiocytoma, a round cell sarcoma, and a synovial sarcoma.

The term “effective amount” of an Aurora kinase inhibitor disclosed herein is a quantity sufficient to, when administered to the subject, including a mammal, for example a human, effect beneficial or desired results, including clinical results. An “effective amount” of a disclosed Aurora kinase inhibitor is an amount which prevents, inhibits, suppresses or reduces the cancer (e.g., as determined by clinical symptoms or the amount of cancer cells) in a subject as compared to a control. Specifically, “treating a subject with a cancer” includes achieving, partially or substantially, one or more of the following: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer (such as tissue or serum components). “Treating a subject with a cancer” also includes prophylactic treatment.

Generally, an effective amount of an disclosed Aurora kinase inhibitor varies depending upon various factors, such as the given drug or compound, the pharmaceutical formulation, the route of administration, the type of disease or disorder, the identity of the subject or host being treated, and the like, but can nevertheless be routinely determined by one skilled in the art. As defined herein, a therapeutically effective amount of a compound of the present invention may be readily determined by one of ordinary skill by routine methods known in the art.

In an embodiment, an effective amount of a disclosed Aurora kinase inhibitor ranges from about 0.1 to about 15 mg/kg body weight, suitably about 1 to about 5 mg/kg body weight, and more suitably, from about 2 to about 3 mg/kg body weight. The skilled artisan will appreciate that certain factors may influence the dosage required to effectively treat a subject suffering from cancer and these factors include, but are not limited to, the severity of the disease or disorder, previous treatments, the general health and/or age of the subject and other diseases present.

Moreover, a “treatment” regime of a subject with an effective amount of a disclosed Aurora kinase inhibitor may consist of a single administration, or alternatively comprise a series of applications. For example, the compound of the present invention may be administered at least once a week. However, in another embodiment, the Aurora kinase inhibitor may be administered to the subject from about one time per week to once daily for a given treatment. The length of the treatment period depends on a variety of factors, such as the severity of the disease, the age of the patient, the concentration and the activity of the Aurora kinase inhibitor, or a combination thereof. It will also be appreciated that the effective dosage of the Aurora kinase inhibitor used for the treatment or prophylaxis may increase or decrease over the course of a particular treatment or prophylaxis regime. Changes in dosage may result and become apparent by standard diagnostic assays known in the art. In some instances, chronic administration may be required.

As used herein, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

In one embodiment, the method of the present invention is a mono-therapy where the pharmaceutical compositions of the invention are administered alone. Accordingly, in this embodiment, the compound of the invention is the only pharmaceutically active ingredient in the pharmaceutical compositions or the only pharmaceutically active ingredient administered to the subject.

In another embodiment, the method of the invention is a co-therapy with one or more of other therapeutically active drugs or therapies known in the art for treating the desired diseases or indications. In one example, one or more other anti-proliferative or anticancer therapies are combined with the compounds of the invention. In another example, the compounds disclosed herein are co-administered with one or more of other anticancer drugs known in the art. Anticancer therapies that may be used in combination with the compound of the invention include surgery, radiotherapy (including, but not limited to, gamma-radiation, neutron beam radiotherapy, electron beam radiotherapy, proton therapy, brachytherapy, and systemic radioactive isotopes) and endocrine therapy. Anticancer agents that may be used in combination with the compounds of the invention include biologic response modifiers (including, but not limited to, interferons, interleukins, and tumor necrosis factor (TNF)), hyperthermia and cryotherapy, agents to attenuate any adverse effects (e.g., antiemetics), and other approved chemotherapeutic drugs (e.g. taxol and analogs thereof).

Examples of anti-cancer agents which can be co-administered with the disclosed Aurora kinase inhibitors include abarelix, alitretinoin, allopurinol, altretamine, amifostine, anakinra, anastrozole, arsenic trioxide, asparaginase, azacitidine, BCG Live, bevacuzimab, bexarotene, bleomycin, bortezomib, busulfan, calusterone, capecitabine, carboplatin, carmustine, celecoxib, cetuximab, chlorambucil, cisplatin, cladribine, clofarabine, cyclophosphamide, cytarabine, dacarbazine, dactinomycin, ctinomycin D, dalteparin sodium, darbepoetin alfa, dasatinib, daunorubicin, daunomycin, decitabine, denileukin, dexrazoxane, docetaxel, doxorubicin, dromostanlone propionate, eculizumab, Elliott's B Solution, epirubicin, epoetin alfa, erlotinib, estramustine, etoposide, exemestane, fentanyl citrate, Filgrastim, floxuridine (intraarterial), fludarabine, fulvestrant, gefitinib, gemcitabine, gemtuzumab ozogamicin, goserelin acetate, histrelin acetate, hydroxyurea, Ibritumomab Tiuxetan, idarubicin, ifosfamide, imatinib mesylate, Interferon alfa-2b, irinotecan, lapatinib ditosylate, lenalidomide, letrozole, leucovorin, Leuprolide Acetate, levamisole, lomustine, CCNU, meclorethamine, nitrogen mustard, megestrol acetate, melphalan, L-PAM, mercaptopurine, 6-MP, mesna, methotrexate, methoxsalen, mitomycin C, mitotane, mitoxantrone, nandrolone phenpropionate, nelarabine, Nofetumomab, Oprelvekin, oprelvekin, oxaliplatin, paclitaxel, palifermin, pamidronate, panitumumab, pegademase, pegaspargase, Pegfilgrastim, Peginterferon alfa-2b, pemetrexed disodium, pentostatin, pipobroman, plicamycin, mithramycin, porfimer sodium, procarbazine, quinacrine, Rasburicase, Rituximab, Sargramostim, sorafenib, streptozocin, sunitinib, talc, tamoxifen, temozolomide, teniposide, VM-26, testolactone, thalidomide, thioguanine, 6-TG, thiotepa, topotecan, toremifene, Tositumomab, trastuzumab, tretinoin, ATRA, Uracil Mustard, valrubicin, vinblastine, vincristine, vinorelbine, vorinostat and zoledronate.

When the compounds of the invention are combined with other anticancer drugs, they can be administered contemperaneously. As used herein, “administered contemporaneously” means that two substances are administered to a subject such that they are both biologically active in the subject at the same time. The exact details of the administration will depend on the pharmacokinetics of the two substances in the presence of each other, and can include administering one substance within a period of time of one another, e.g., 24 hours of administration of the other, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances. Alternatively, the two agents can be administered separately, such that only one is biologically active in the subject at the same time.

The compounds of the invention can be administered to a patient in a variety of forms depending on the selected route of administration, as will be understood by those skilled in the art. The compounds of the invention may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time. Oral and parenteral administration are most commonly used.

The compounds of the invention can be suitably formulated into pharmaceutical compositions for administration to a subject. The pharmaceutical compositions of the invention optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

Typically, for oral therapeutic administration, a compound of the invention may be incorporated with excipient and used in the form of ingestible tablets, buccal tablets, troches, capsules, elixirs, suspensions, syrups, wafers, and the like.

Typically for parenteral administration, solutions of a compound of the invention can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound of the invention for the extemporaneous preparation of sterile injectable solutions or dispersions.

For nasal administration, the disclosed Aurora kinase inhibitors can be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

For buccal or sublingual administration, the disclosed Aurora kinase inhibitors can be formulated with a carrier such as sugar, acacia, tragacanth, or gelatin and glycerine, as tablets, lozenges or pastilles.

For rectal administration, the disclosed Aurora kinase inhibitors can be formulated in the form of suppositories containing a conventional suppository base such as cocoa butter.

The disclosed Aurora kinase inhibitors can be formulated alone or for contemporaneous administration with other agents for treating cancer. Therefore, in another aspect, a pharmaceutical composition of the invention comprises a pharmaceutically acceptable carrier or diluent, an Aurora kinase inhibitor disclosed herein or a pharmaceutically acceptable salt thereof and another anti-cancer agent.

The invention is illustrated by the following examples which are not intended to be limiting in any way.

The following abbreviations have the indicated meanings:

| Abbrevi- | |
|---|---|
| ation | Meaning |
| Boc | tert-butoxy carbonyl or t-butoxy carbonyl |
| (Boc)₂O | di-tert-butyl dicarbonate |
| Cbz | Benzyloxycarbonyl |
| CbzCl | Benzyl chloroformate |
| DAST | diethylaminosulfur trifluoride |
| DBU | 1,8-diazabicyclo[5.4.0]undec-7-ene |
| DCC | N,N′-dicyclohexylcarbodiimide |
| DCU | N,N′-dicyclohexylurea |
| DIAD | diisopropyl azodicarboxylate |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| 2,4-DNP | 2,4-dinitrophenylhydrazine |
| DPTBS | Diphenyl-t-butylsilyl |
| EDC•HCl, | 1-[3-(dimethylamino)propyl]-3-ethylcarbodiimide |
| EDCI | hydrochloride |
| Equiv | equivalents |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour(s) |
| HOBt | 1-hydroxybenzotriazole |
| HATU | 2-(7-Aza-1H-benzotriazole-1-yl)-1,1,3,3-tetramethyluronium |
| | hexafluorophosphate |
| HBTU | 2-1H-Benzotriazol-1-yl)-1,1,3,3-tetramethyluronium |
| | hexafluorophosphate |
| KHMDS | potassium hexamethyldisilazane |
| LAH or | lithium aluminum hydride |
| LiAlH₄ | |
| LC-MS | liquid chromatography-mass spectroscopy |
| LHMDS | lithium hexamethyldisilazane |
| Me | methyl |
| MsCl | methanesulfonyl chloride |
| Min | minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMM | N-methylmorpholine |
| NMP | N-methylpyrrolidinone |
| Pd₂(dba)₃ | tris(dibenzylideneacetone)dipalladium(0) |
| PE | petroleum ether |
| Quant | quantitative yield |
| Satd | saturated |
| SOCl₂ | thionyl chloride |
| SPA | scintillation proximity assay |
| SPE | solid phase extraction |
| TBAF | tetrabutylammonium fluoride |
| TBS | t-butyldimethylsilyl |
| TBDPS | t-butyldiphenylsilyl |
| TBSCl | t-butyldimethylsilyl chloride |
| TBDPSCl | t-butyldiphenylsilyl chloride |
| TEA | triethylamine or Et₃N |
| TEMPO | 2,2,6,6-tetramethyl-1-piperidinyloxy free radical |
| Teoc | 1-[2-(trimethylsilyl)ethoxycarbonyloxy]- |
| Teoc-OSu | 1-[2-(trimethylsilyl)ethoxycarbonyloxy]pyrrolidin-2,5-dione |
| TFA | trifluoroacetic acid |
| Tlc, TLC | thin layer chromatography |
| TMS | trimethylsilyl |
| TMSCl | chlorotrimethylsilane or trimethylsilyl chloride |
| t_{R} | retention time |
| TsOH | p-toluenesulfonic acid |

### General Description of Synthetic Methods

Compounds of the Formula I can be prepared by several processes. In the discussion below s, X, Y, Ring A, R¹, R², R^{3a}, R^{3b}, R^{3c} and R⁴ have the meanings indicated above unless otherwise noted. In cases where the synthetic intermediates and final products of Formula I described below contain potentially reactive functional groups, for example amino, hydroxyl, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). Such protecting group manipulations are assumed in the discussion below and not described explicitly. Generally, reagents in the reaction schemes are used in equimolar amounts; however, in certain cases it may be desirable to use an excess of one reagent to drive a reaction to completion. This is especially the case when the excess reagent can be readily removed by evaporation or extraction. Bases employed to neutralize HCl in reaction mixtures are generally used in slight to substantial excess (1.05-5 equivalents).

In the first process, a compound of Formula I can be prepared by Suzuki or Stille reactions of an intermediate of Formula XIV, wherein G1 is Br, I, Cl or OTf, with a reagent of Formula XV, wherein G2 is boronic acid, boronic ester or alkyltin. Reagents of Formula XV are either commercially available or can be prepared readily from commercially available precursors based on literature precedents. G1 and G2 can be reversed if so desired.

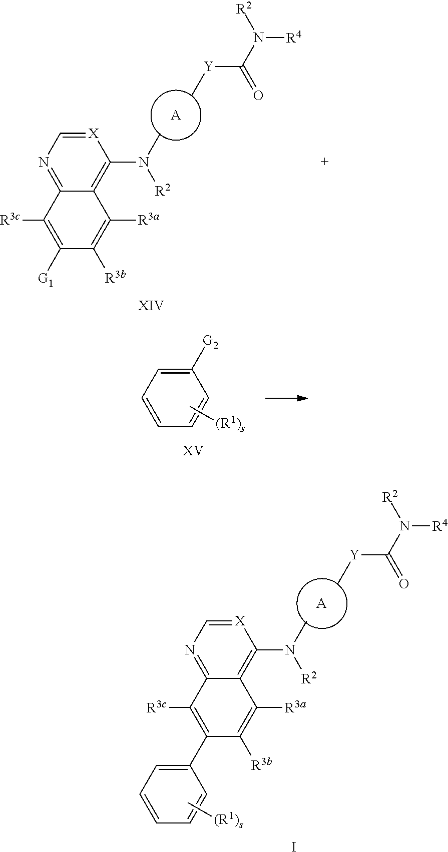

Intermediates of Formula XIV can be prepared by amide coupling between an acid of Formula XVI with an amine of Formula XVII.

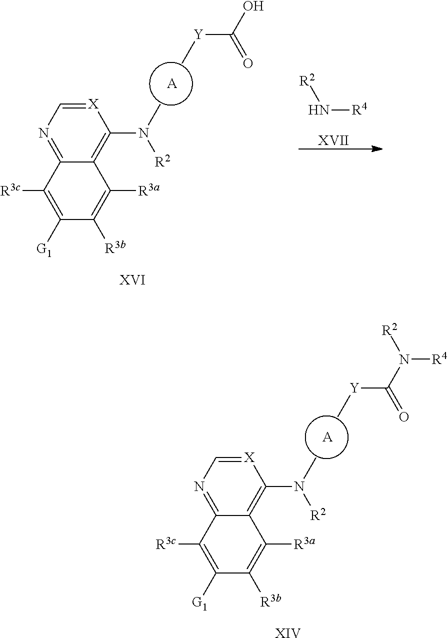

Intermediates of Formula XVI can be prepared by displacement of G3 in an intermediate of Formula XVIII, wherein G3 is Cl, Br, I or OTf, with an amine of Formula XIX.

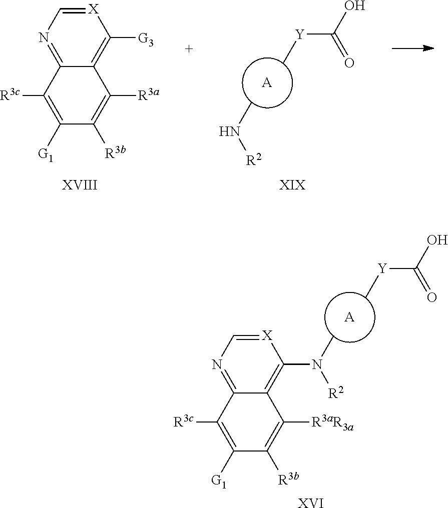

Alternatively, Intermediates of Formula XIV can be prepared by displacement of G3 in an intermediate of Formula XVIII, wherein G3 is Cl, Br, I or OTf, with an amine of Formula XIXa.

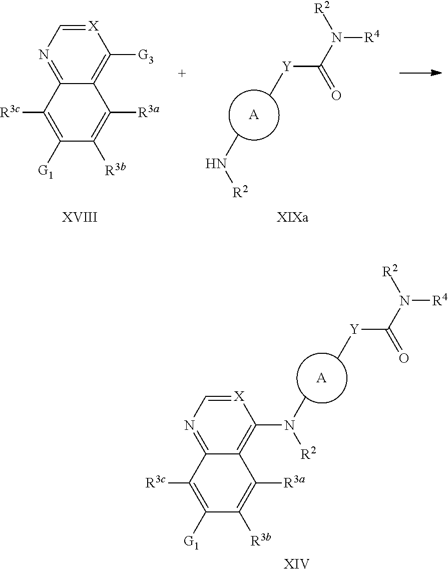

Intermediates of Formula XIXa in turn can be prepared by reaction of an acid of Formula XIX with an amine of Formula XVII.

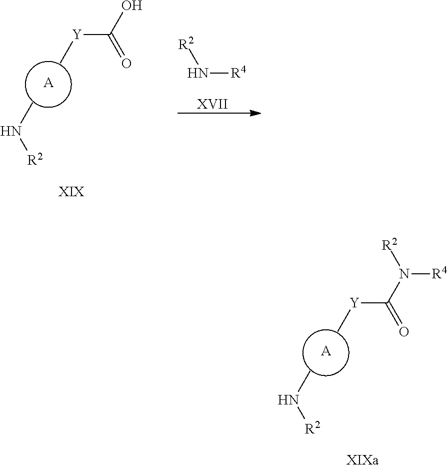

Intermediates of Formula XVIII can be prepared from commercially available alcohols of Formula XX using POCl3, POBr3, or Tf2O etc.

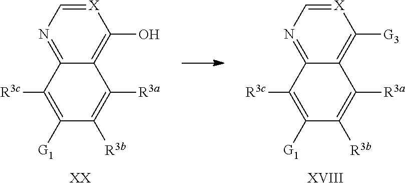

Or alcohols of Formula XX wherein X═N can be prepared from anilines XXa (Baker et al. J. Org. Chem. 1952, 17, 141 and Zheng et al. WO2005042498):

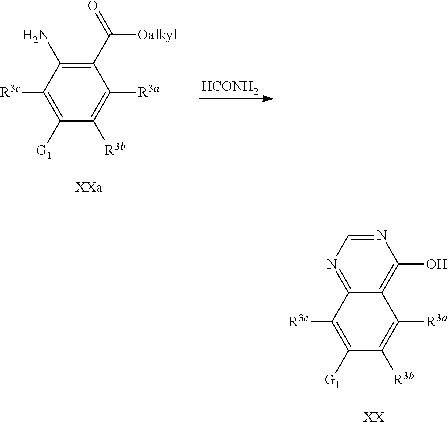

Or alcohol of Formula XX wherein X═CR_{A} can be prepared from anilines XXb (Baker et al. J. Med. Chem. 1972, 15, 235 and Tsou at al. J. Med. Chem. 2005, 48, 1107):

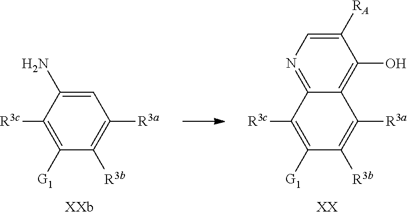

Intermediates of Formula XIX can be prepared according to literature. However, for illustrative purpose, when A is a pyrazole ring and Y═CH2, a compound of formula XX may be prepared according to the following scheme (Dickinson et al J. Org. Chem. 1964, 29, 1915 and Mortlock et al. J. Med. Chem. 2007, 50, 2213):

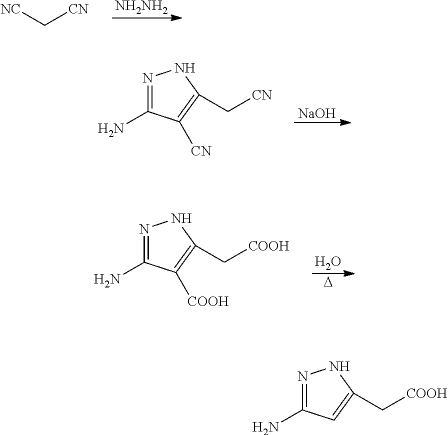

In the second process, a compound of Formula I can be prepared by displacement of G3 in an intermediate of Formula XIVa, wherein G3 is Cl, Br, I or OTf, with an amine of Formula XIXa.

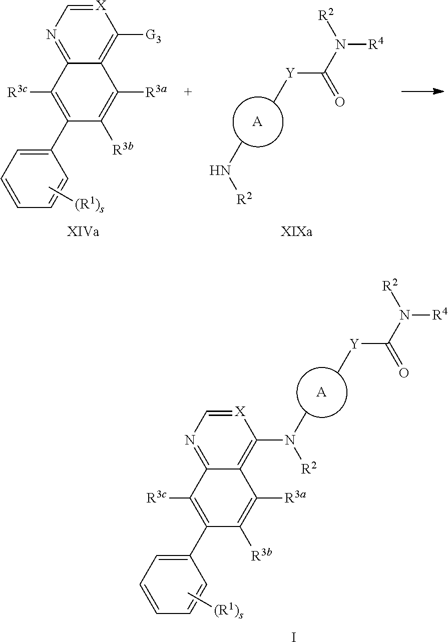

Intermediates of Formula XIVa can be prepared from alcohols of Formula XVIIIa using POCl3, POBr3 or Tf2O etc.

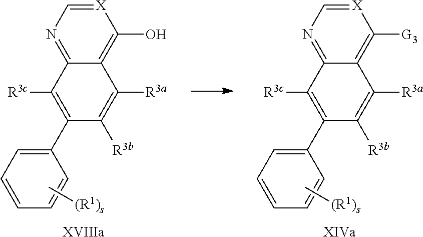

Intermediates of Formula XVIIIa can be prepared by Suzuki or Stille reactions of an intermediate of Formula XVIII, wherein G1 is Br, I, Cl or OTf, with a reagent of Formula XV, wherein G2 is boronic acid, boronic ester or alkyltin. Reagents of Formula XV are either commercially available or can be prepared readily from commercially available precursors based on literature precedents. G1 and G2 can be reversed if so desired.

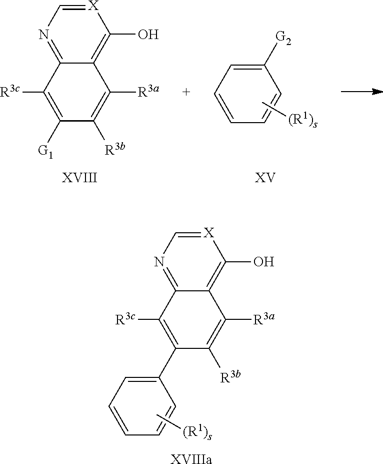

In the third process, a compound of Formula I can be prepared by amide coupling between an acid of Formula XIVb and an amine of Formula XVII.

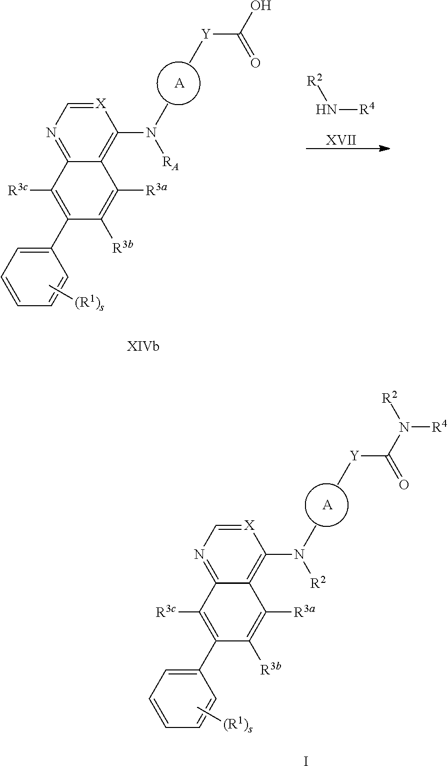

Intermediates of Formula XIVb can be prepared by displacement of G3 in an intermediate of Formula XIVa, wherein G3 is Cl, Br, I or OTf, with an amine of Formula XIX.

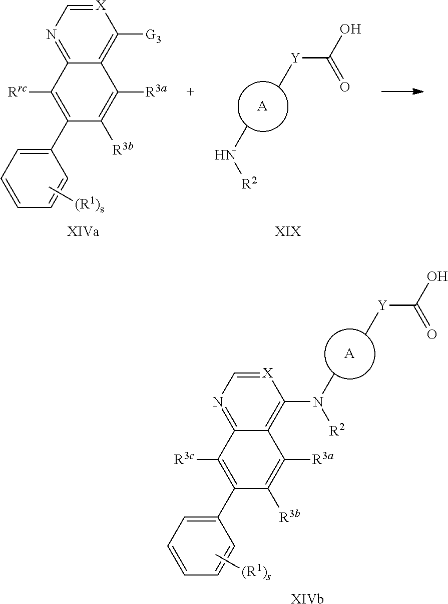

### Purification Methods

Compounds of the invention may be purified by high pressure liquid chromatography (prep HPLC). Unless otherwise specified, prep HPLC refers to preparative reverse phase HPLC on a C-18 column eluted with a water/acetonitrile gradient containing 0.01% TFA run on a Gilson 215 system.

### LC-MS Methods

Method 1 [LC-MS (3 min)]
Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time (min) | A % | B % |
|---|---|---|
| 0.0 | 90 | 10 |
| 2.0 | 10 | 90 |
| 2.4 | 10 | 90 |
| 2.5 | 90 | 10 |
| 3.0 | 90 | 10 |

### Method 2 (10-80)

| | |
|---|---|
| Column | YMC-PACK ODS-AQ, 50 × 2.0 mm 5 μm |
| Mobile | A: water (4 L) + TFA (1.5 mL)) |
| Phase | B: acetonitrile (4 L) + TFA (0.75 mL)) |

| | | |
|---|---|---|
| TIME(min) | A % | B % |
| 0 | 90 | 10 |
| 2.2 | 20 | 80 |
| 2.5 | 20 | 80 |

| | |
|---|---|
| Flow Rate | 1 mL/min |
| Wavelength | UV 220 nm |
| Oven Temp | 50° C |
| MS | ESI |
| ionization | |

### Method 3 (30-90)

| | |
|---|---|
| Column | YMC-PACK ODS-AQ , 50 × 2.0 mm 5 μm |
| Mobile | A: water (4 L) + TFA (1.5 mL)) |
| Phase | B: acetonitrile (4 L) + TFA (0.75 mL)) |

| | | |
|---|---|---|
| TIME(min) | A % | B % |
| 0 | 70 | 30 |
| 2.2 | 10 | 90 |
| 2.5 | 10 | 90 |

| | |
|---|---|
| Flow Rate | 1 mL/min |
| Wavelength | UV220 |
| Oven Temp | 50° C |
| MS | ESI |
| ionization | |

### EXAMPLE 1

### 2-(3-(7-bromoquinazolin-4-ylamino)-1H-pyrazol-5-yl)-N-(3-fluorophenyl)acetamide

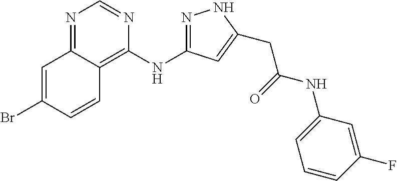

### Step 1. 3-amino-5-(carboxymethyl)-1H-pyrazole-4-carboxylic acid

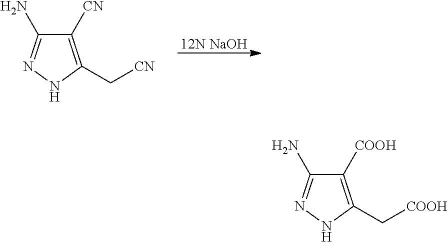

A suspension of 3-amino-5-(cyanomethyl)-1H-pyrazole-4-carbonitrile (2.0 g, 13.6 mmol) in an aqueous solution of sodium hydroxide (12 M, 20 mL) was heated to reflux overnight. The resultant solution was cooled in an ice/water bath and then made acidic (pH˜3) by the addition of concentrated hydrochloric acid. The resultant solid was filtered, washed with water, and then dried under lamp to give crude 3-amino-5-carboxymethyl-1H-pyrazole-4-carboxylic acid (2.7 g).

### Step 2. 2-(3-amino-1H-pyrazol-5-yl)acetic acid

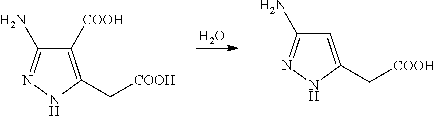

3-Amino-5-carboxymethyl-1H-pyrazole-4-carboxylic acid (1.7 g, crude, 8.45 mmol) was suspended in water (30 mL) and heated to reflux overnight. The mixture was allowed to cool to room temperature and then filtered. The filtrate was evaporated to dryness, and the residue was triturated with ethyl acetate and then dried under lamp to give the desired 2-(3-amino-1H-pyrazol-5-yl)acetic acid (0.9 g, 76%). ¹H NMR (DMSO-d₆) δ 5.60˜8.50 (br s, 4H), 5.25 (s, 1H), 3.34 (s, 2H).

### Step 3. 7-bromo-4-chloroquinazoline

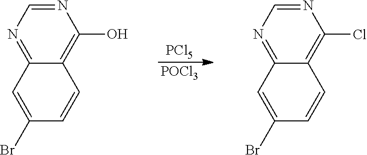

To a solution of 7-bromoquinazolin-4-ol (2.88 g, 12.8 mmol) in POCl₃ (30 mL) was added PCl₅ (4 g, 19.2 mmol). The mixture was stirred at reflux overnight. After cooling to room temperature, the mixture was concentrated. The residue was dissolved in CH₂Cl₂ and treated with saturated aqueous NaHCO₃ solution. A precipitate that formed was filtered off from the mixture. The organic layer was washed with brine, dried and concentrated to give 7-bromo-4-chloroquinazoline (3 g, 97%). ¹H NMR (DMSO-d₆) δ 8.36 (s, 1H), 8.01 (d, J=8.40 Hz, 1H), 7.88 (d, J=1.60 Hz, 1H), 7.69 (dd, J=8.80 Hz, 1.60 Hz, 1H).

### Step 4. 2-(3-(7-bromoquinazolin-4-ylamino)-1H-pyrazol-5-yl)-N-(3-fluorophenyl)acetamide

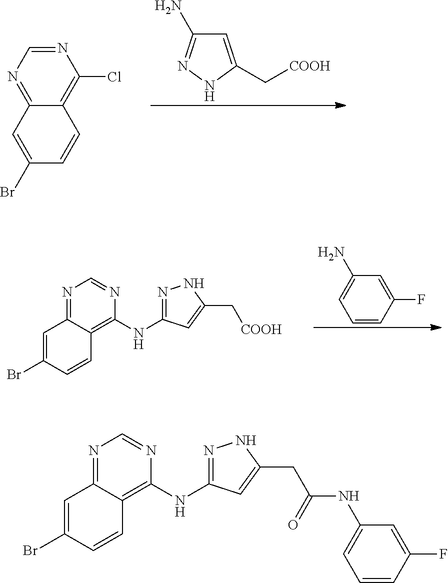

A solution of hydrogen chloride in dioxane (4N, 1.78 mL, 7.1 mmol) was added to a mixture of 7-bromo-4-chloroquinazoline (2.58 g, 10.6 mmol) and 2-(3-amino-1H-pyrazol-5-yl)acetic acid (1 g, 7.1 mmol) in dimethylacetamide (50 mL), and the mixture was stirred overnight at room temperature. The mixture was then treated with EDCI (2.8 g, 14.2 mmol), HOBt (2.23 g, 14.2 mmol), 3-fluorobenzenamine (3.15 g, 28.4 mmol) and DIEA (9.2 g, 71 mmol) at 0° C. After stirring overnight, the resultant solution was treated with water and ethyl acetate. The layers were separated, and the organic layer was washed with water and brine, dried over Na₂SO₄, and concentrated to give a brown solid. The solid was washed with dichloromethane and then recrystallized from methanol to give 2-(3-(7-bromoquinazolin-4-ylamino)-1H-pyrazol-5-yl)-N-(3-fluorophenyl)acetamide (1 g, yield 32.5%). LC/MS m/z 440 (M⁺). ¹H NMR (DMSO-d₆) δ 8.56 (s, 2H), 7.93 (s, 1H), 7.71 (d, J=8.40 Hz, 1H), 7.60 (d, J=11.60 Hz, 1H), 7.32 (m, 2H), 6.87 (t, J=7.60 Hz, 1H), 6.75 (m, 1H), 3.73 (s, 2H).

### EXAMPLE 2

### N-(3-fluorophenyl)-2-(3-(7-(4-(4-(2-hydroxyethyl)piperazine-1-carbonyl)phenyl)quinazolin-4-ylamino)-1H-pyrazol-5-yl)acetamide (Compound 1)

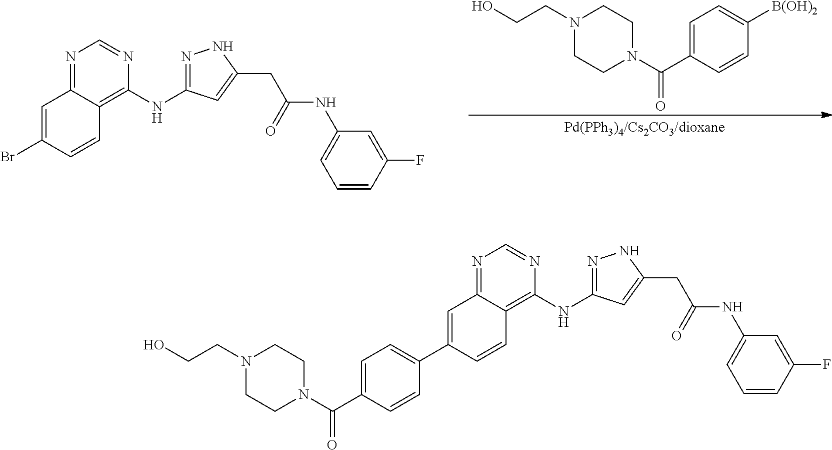

To a mixture of 2-(3-(7-bromoquinazolin-4-ylamino)-1H-pyrazol-5-yl)-N-(3-fluorophenyl)acetamide (500 mg, 1.1 mmol), tetrakis-(triphenylphosphine)palladium(0) (100 mg, 0.09 mmol), 4-(4-(2-hydroxyethyl)piperazine-1-carbonyl)phenylboronic acid (630 mg, 2.3 mmol) in 1,4-dioxane (50 ml) was added an aqueous solution of cesium carbonate (2M, 6.8 mL, 13.6 mmol). The above mixture was heated to 120° C. and stirred overnight under N₂. After cooling to room temperature, the reaction mixture was filtered. The filtrate was concentrated and purified by preparative HPLC to give N-(3-fluorophenyl)-2-(3-(7-(4-(4-(2-hydroxyethyl)piperazine-1-carbonyl)phenyl)quinazolin-4-ylamino)-1H-pyrazol-5-yl)acetamide (42.51 mg, yield 6.3%). LC/MS m/z 595 (M+1). ¹H NMR (MeOD) δ 8.86 (s, 1H), 8.68 (d, J=8.80 Hz, 1H), 8.16 (d, J=8.80 Hz, 1H), 8.10 (s, 1H), 7.97 (d, J=8.00 Hz, 2H), 7.70 (d, J=8.40 Hz, 2H), 7.58 (d, J=11.20 Hz, 1H), 7.28-7.32 (m, 2H), 6.83 (m, 2H), 3.91-4.30 (m, 2H), 3.91 (t, J=5.20 Hz, 2H), 3.87 (s, 2H), 3.35-3.70 (m, 4H), 3.30-3.35 (m, 4H).

This compound was also prepared in gram quantity scale by following procedure:

Step 1. Preparation of Compound 2

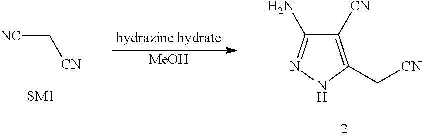

Material Resource (Reaction)

| | Material | Amount | | Equiv. | | W/W | Vol/W |
|---|---|---|---|---|---|---|---|
| 1 | Malononitrile | 500 | g | 1.0 | eq | | |
| 2 | N₂H₄•H₂O | 240 | g | 0.55 | eq | 0.48 | |
| 3 | Methanol | 1.5 | L | | | | 3.0 |
| | Compound 2 | 330 | g | | | 0.66 | |

Procedure Description:

1.5 L of MeOH was charged into a three-necked flask (5 L). SM1 (500 g, 7.58 mol) and N₂H₄.H₂O (60 g, 1.02 mol) were charged. The solution was heated to reflux at between 64-68° C. and then removed the oil bath. N₂H₄.H₂O (180 g, 3.06 mol, 85%) was re-added to reaction solution quickly at the rate of maintaining reaction solution refluxing (about 15 min). The mixture was refluxed for additional 15 min, then cooled down to 0-5° C. quickly and stirred for 2 h at the same temperature. Solid was collected by filtration, washed with 200 mL of water and followed by 50 mL of pre-cooled MeOH. Obtained 330 g of wet compound 2 was used to synthesize compound 3 directly.

Step 2. Preparation of Compound 3

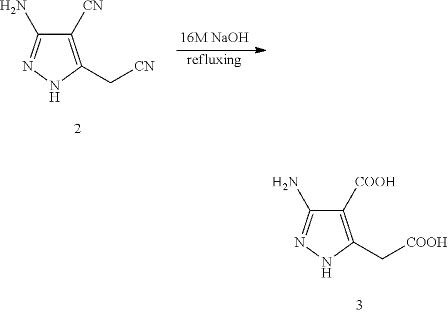

Material Resource (Reaction)

| | Material | Amount | | Equiv. | W/W | Vol/W |
|---|---|---|---|---|---|---|
| 1 | Compound 2 | 330 | g | 1.0 eq | | |
| 2 | NaOH (16M aq. Solution) | 1350 | g | | 4.0 | |
| 3 | conc. HCl | 1.2 | L | | | 3.6 |
| | Compound 3 | 460 | g | | 1.4 | |

Procedure Description:

NaOH (528 g, 13.2 mol) was added to water (825 mL), and the suspension was heated to 98-103° C. to give a resulting a clear solution (16M NaOH aqueous). Wet compound 2 (330 g) was added to the solution in one portion and stirred for 16 h at 98-105° C. HPLC indicated that material compound 2 was consumed completely. The solution was cooled down to 0-5° C., and conc. HCl (1200 mL) was added drop-wise at this temperature to adjust PH=3-4. Precipitate was collected by filtration, and washed with 200 mL of water. Wet product (above 460 g) with 96.5% HPLC purity was obtained and then used directly to next step without any further purification.

Step 3. Preparation of Compound 4

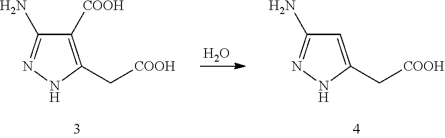

Material Resource (Reaction)

| | Material | Amount | | Equiv. | W/W | Vol/W |
|---|---|---|---|---|---|---|
| 1 | Compound 3 | 460 | g | 1.0 eq | | |
| 2 | Water | 2 | L | | | 4.35 |
| 4 | Compound 4 | 192 | g | | 0.41 | |

Procedure Description:

Above wet compound 3 (460 g, 2.48 mol) was suspended into 2 L of water (5 L flask) at 18-20° C. The suspension was heated to reflux slowly over 2.5 h and then a clear solution was obtained. The solution was refluxed for additional 2.5 h at 95-100° C. The reaction was indicated by HPLC to complete conversion. Water was removed under reduced pressure to remain about 150 mL of suspension. Suspension was cooled down to 15-20° C., and stirred for 0.5 h. Then the mixture was filtered and washed with 50 mL of water. Solid was dried under vacuum at 50° C. for 12 h to give 192 g of compound 4 with 97.2% HPLC purity in 35.9% overall yield corresponding to 500 g of SM1. ¹H NMR (DMSO-d₆) δ 5.22 (s, 1H), 3.35 (s, 2H).

Step 4. Preparation of Compound 6

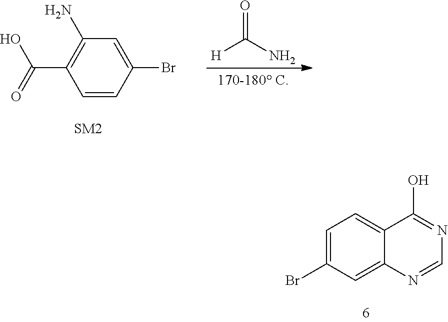

Material Resource (Reaction)

| | Material | Amount | | Equiv. | W/W | Vol/W |
|---|---|---|---|---|---|---|
| 1 | 2-amino-4- | 300 | g | 1.0 eq | | |
| | bromobenzoic acid | | | | | |
| 2 | Formamide | 3 | L | | | 10.0 |
| 3 | Acetone | 500 | mL | | | 1.67 |
| 4 | Compound 6 | 240 | g | | 0.8 | |

Procedure Description:

To a solvent of formamide (3 L) was added SM2 (300 g, 1.39 mol, 1.0 eq.) at 15-18° C. The suspension was heated to 170-180° C. for 3 h and the suspension turned to clear solution after 2 hours later. HPLC analysis indicated that material was consumed completely. The reaction mixture was cooled down to 20-25° C. slowly and stirred for additional 30 min at this temperature. The precipitate was filtered, and washed with 500 mL of acetone. Solid was dried under vacuum at 60° C. (12 h) to give 240 g of compound 6 with 99.1% HPLC purity in 76.8% yield.

Step 5. Preparation of Compound 7

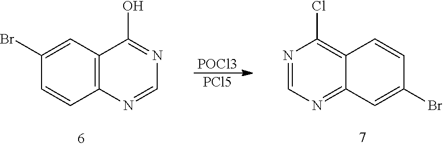

Material Resource (Reaction)

| | Material | Amount | | Equiv. | W/W | Vol/W |
|---|---|---|---|---|---|---|
| 1 | Compound 6 | 110 | g | 1.0 | | |
| 2 | POCl₃ | 1.1 | kg | 14.7 | 10.0 | |
| 3 | PCl₅ | 147 | g | 1.45 | 1.33 | |
| 4 | CH₂Cl₂ | 2.0 | L | | | 18.8 |
| 5 | Sat. NaHCO₃ | 3.0 | L | | | 27.2 |
| | Compound 7 | 100 | g | | 0.9 | |

Procedure Description:

To a mixture of compound 6 (110 g, 0.489 mol, 1.0 eq.) and POCl₃ (1100 g, 7.19 mol) was added PCl₅ (146.6 g, 0.705 mol) at 15-18° C. The suspension was heated to reflux at 105-107° C. for 4 h (resulting a clear solution about two hours later), and compound 6 was consumed completely showed by HPLC. The solution was cooled down to 55-60° C., and concentrated under reduced pressure to remove POCl₃ at 55-65° C. The residue was dissolved in 2000 mL of CH₂Cl₂, and this solution was added drop-wise to 3.0 L of saturated NaHCO₃ aqueous solution at 15-20° C. under vigorous stirring (pH=8). Organic layer was separated out and washed with 1.2 L of brine twice, and then dried over anhydrous Na₂SO₄. The organic phase was concentrated, and dried under oven at 50° C. (12 h) to give 100 g compound 7 with 95.2% HPLC purity in 84.1% isolated yield. ¹H NMR (CDCl₃) δ 8.91 (s, 1H), 8.21 (s, 1H), 8.10 (d, J=8.40 Hz, 1H), 7.77 (d, J=8.80 Hz, 1H).

Step 6. Preparation of Compound 9-1

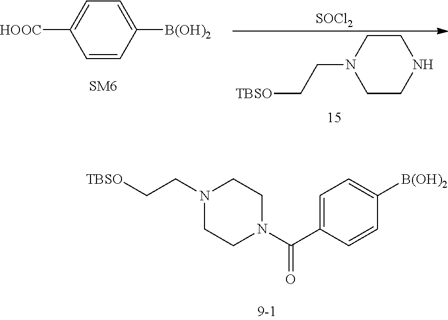

Material Resource (Reaction)

| | Material | Amount | | Equiv. | W/W | Vol/W |
|---|---|---|---|---|---|---|
| 1 | 4-boronobenzoic acid | 53 | g | 1.0 | | |
| 2 | Compound 15 | 93.5 | g | 1.2 | | |
| 3 | SOCl2 | 800 | mL | | | 15.0 |
| 4 | Dichloromethane | 300 | mL | | | 5.7 |
| 5 | THF | 600 | mL | | | 11.3 |
| 6 | TEA | 96.7 | g | 3.0 | 1.8 | |
| 7 | Aq. NaHCO3 (5%) | 1.0 | L | | | 18.8 |
| 8 | MTBE | 1.6 | L | | | 30.0 |
| | Compound 9-1 | 103 | g | | 1.9 | |

Procedure Description:

SM6 (53 g, 0.319 mol, 1.0 eq.) was suspended in 800 mL of SOCl₂, and 6 mL of DMF (HPLC level) was added at 20-25° C. The suspension was heated to reflux for 3 h at 76-78° C. SOCl₂ was removed by distillation, and DCM (150 mL×2) was added to replace the excess SOCl₂ completely. The residue was dissolved in 600 mL of anhydrous THF, and this solution was added drop-wise into a suspending mixture of TEA (96.7 g, 0.957 mol, 3.0 eq.) and compound 15 (93.5 g, 0.383 mol, 1.2 eq.) in 400 mL of anhydrous THF at 0-5° C. over 30 min. Then the suspension was warmed and stirred at 15-22° C. for 1.5 h. NaHCO₃ aqueous solution (1 L, 5%) was added to reaction mixture. The organic layer was separated out and the water phase was back-extracted by MTBE (800 mL×2). Combined organic layers were washed with brine (1.2 L×2), and dried over anhydrous Na₂SO₄. The mixture was concentrated under reduced pressure to give brown oil. This brown oil was re-dissolved in 1 L of MTBE, and washed with water (500 mL×2), dried over anhydrous Na₂SO₄. Concentrated under reduced pressure to a less volume (about 150 mL), and 500 mL of n-Heptane was added and re-concentrated to solidify compound 9-1. The solid was dried under vacuum at 35° C. (12 h) to give 103 g of compound 9-1 with 98.3% HPLC purity in 75.4% isolated yield. ¹H NMR (CD₃OD) δ 7.69 (d, J=7.20 Hz, 2H), 7.28 (d, J=8.0 Hz, 2H), 3.60-3.80 (m, 4H), 3.30-3.40 (m, 2H), 2.40-2.65 (m, 6H), 0.82 (s, 9H), 0.0 (m, 6H).

Step 7. Preparation of Compound 10

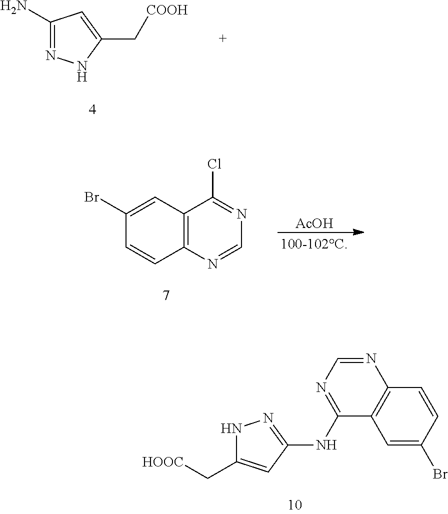

Material Resource (Reaction)

| | Material | Amount | | Equiv. | W/W | Vol/W |
|---|---|---|---|---|---|---|
| 1 | Compound 7 | 96.0 | g | 1.0 | | |
| 2 | Compound 4 | 83.5 | g | 1.5 | 0.87 | |
| 3 | Acetic acid | 960 | mL | | | 10.0 |
| 4 | Ethyl acetate | 100 | mL | | | 1.0 |
| | Compound 10 | 136 | g | | 1.4 | |

Procedure Description:

Compound 7 (96 g, 0.395 mol, 1.0 eq.) and compound 4 (83.5 g, 0.592 mol, 1.5 eq.) were suspended in 960 mL of AcOH at 18-20° C. The suspension was heated to 100-102° C., and stirred for 2.5 h at this temperature. The suspension was cooled down to 18-20° C., and filtered. Filtrate cake was washed with 50 mL of AcOH, followed by 100 mL of EtOAc. Solid was collected and dried under vacuum at 50° C. (12 h) to give 136 g of compound 10 with 96.5% HPLC purity in 98.9% yield.

Step 8. Preparation of Compound 11

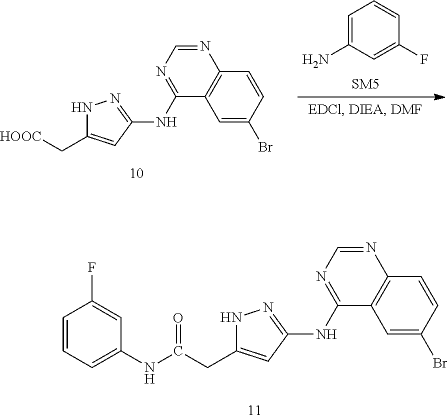

Material Resource (Reaction)

| | Material | Amount | | Equiv. | W/W | Vol/W |
|---|---|---|---|---|---|---|
| 1 | Compound 10 | 102.0 | g | 1.0 | | |
| 2 | 2-fluorobenzene amine | 49 | g | 1.5 | | |
| 3 | EDCI | 68 | g | 1.2 | | |
| 4 | 2-Hydroxy-pyridine-1- | 39.1 | g | 1.2 | | |
| | oxide | | | | | |
| 5 | DIEA | 45.6 | g | 1.2 | | |
| | DMF | 1000 | mL | | | |
| | Compound 11 | 81 | g | | 0.8 | |

Procedure Description:

1000 mL of anhydrous DMF was charged into flask. Then all of reagents were added subsequently with the order of compound 10 (102 g, 0.293 mol, 1.0 eq.), EDCI (68 g, 0.354 mol, 1.2 eq.), 2-Hydroxy-pyridine-1-oxide (39.1 g, 0.352 mol, 1.2 eq.), DIEA (45.6 g, 0.353 mol, 1.2 eq.) and SM5 (49 g, 0.441 mol, 1.5 eq.) at 20-22° C. The solution was stirred for 16 h at 20-25° C. (dark red color). HPLC indicated that the material compound 10 has been consumed completely. 800 mL of water was added to reaction mixture at 25-35° C. and stirred for 30 min. Then the precipitate was filtered and filtrate cake was washed with 500 mL of MeOH. The filtrate cake was collected and dried under oven at 50° C. for 12 h to give 81 g of compound 11 with 90.6% HPLC purity in 62.6% yield.

Step 9. Preparation of Compound 15

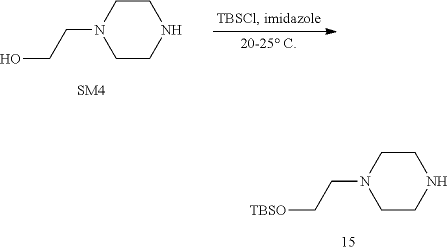

Material Resource (Reaction)

| | Material | Amount | | Equiv. | WAV | Vol/W |
|---|---|---|---|---|---|---|
| 1 | SM4 | 104 | g | 1.0 | | |
| 2 | TBSCl | 180 | g | 1.5 | 1.7 | |
| 3 | Imidazole | 81.6 | g | 1.5 | 0.8 | |
| 4 | THF | 1.2 | L | | | 11.5 |
| | Compound 15 | 103 | g | | 1.0 | |

Procedure Description:

TBSCl (180 g, 1.2 mol, 1.5 eq.) was dissolved in 500 mL of anhydrous THF. This solution was added to a solution of imidazole (81.6 g, 1.2 mol, 1.5 eq) and SM4 (104 g, 0.8 mol, 1.0 eq) in 700 mL of anhydrous THF at 0-15° C. over 30 min. The suspension was warmed and stirred for 20 h at 20-25° C. HPLC (ELSD) analysis indicated that SM4 was consumed completely. 800 mL of water was added to reaction mixture, and this mixture was concentrated (reserved most of water). Additional 800 mL of water was added, and this water phase was extracted by mixed solvent of DCM/isopropanol (3/1, v/v, 1.2 L) two times, and then dried over MgSO₄. The mixture was filtered, and concentrated to give yellow oil. 800 mL of n-heptane was added to the yellow oil. And the mixture was heated to result a clear solution at 55-60° C. The solution was cooled down to 0° C., and stirred for 1 h. Then the mixture was filtered, and washed with 100 mL of pre-cooled n-heptane. Solid was dried under oil pump to give 103 g of white product with 100% HPLC purity in 52.7% yield. ¹H NMR (CDCl₃) δ 3.68 (t, J=5.60 Hz, 2H), 3.15-3.17 (m, 4H), 2.79-2.82 (m, 4H), 2.54 (t, J=5.60 Hz, 2H), 0.83 (s, 9H), 0.0 (s, 6H).

Step 10. Preparation of Compound 14

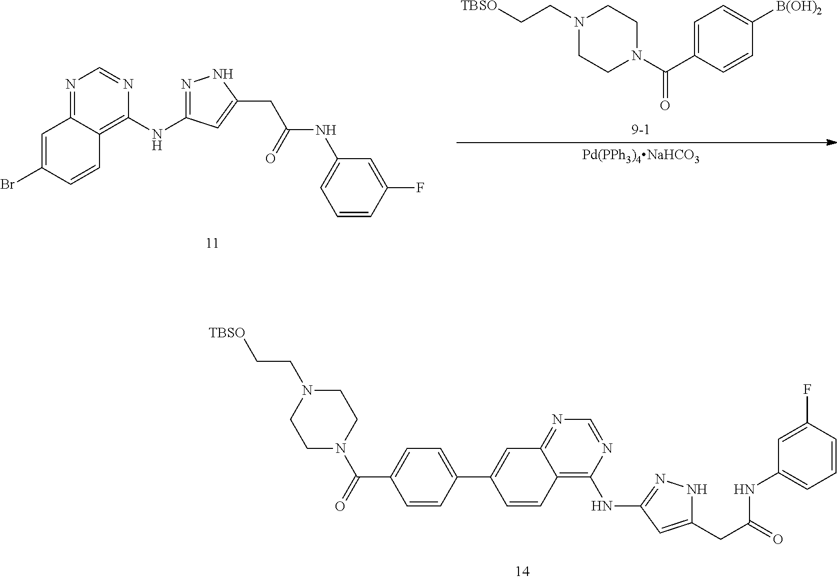

Material Resource (Reaction)

| | Material | Amount | | Equiv. | W/W | Vol/W |
|---|---|---|---|---|---|---|
| 1 | Compound 11 | 48.7 | g | 1.0 | | |
| 2 | Compound 9-1 | 55.7 | g | 1.3 | | |
| 3 | Pd(PPh3)4 | 10 | g | | 0.2 | |
| 4 | NaHCO3 | 46.7 | g | 5.0 | 0.95 | |
| 5 | Dioxane | 1 | L | | | 20.5 |
| | Compound 14 | 35 | g | | 0.71 | |

Procedure Description:

To a suspension of compound 11 (48.72 g, 0.11 mol, 1.0 eq.) and compound 9-1 (55.73 g, 0.14 mol, 1.3 eq.) in dioxane (1 L, 20×) and water (100 mL, 2×) was added NaHCO3 (46.77 g, 0.56 mol, 5.0 eq.) and Pd(PPh3)4 (10.0 g, 20.0% wt.). Then the mixture was heated to 91° C. and stirred at this temperature for 28 h. HPLC analysis indicated that the compound 11 has been consumed completely. To the mixture was added mixed solvent of EtOAc/i-PrOH (v/v=3:1, 500 mL) and brine (500 mL). The organic layer was separated out. The water phase was back-extracted by mixed solvent of EtOAc/i-PrOH (v/v=3:1, 250 mL) one time. Then the organic layer was dried over MgSO₄. The mixture was filtered and concentrated to give black solid. The solid was re-dissolved in DCM (650 mL). And the mixture was dried over MgSO4. The mixture was filtered, and concentrated to give black solid (130 g). The black solid was quickly gone through a silica gel plug (700 g) eluting with 1 L of dichloromethane and then washed with 4 L of THF. Then desired fraction was evaporated to give 72 g of orange solid. The solid was re-slurried with 700 mL of mixed solvent of THF/MeOH (1:1, v/v). The precipitate was filtered and dried under oven (45-50° C.) to afford 35 g of desired product with 94.7% HPLC purity in 44.8% isolated yield, as an off-white solid.

Step 11. Preparation of Final Compound

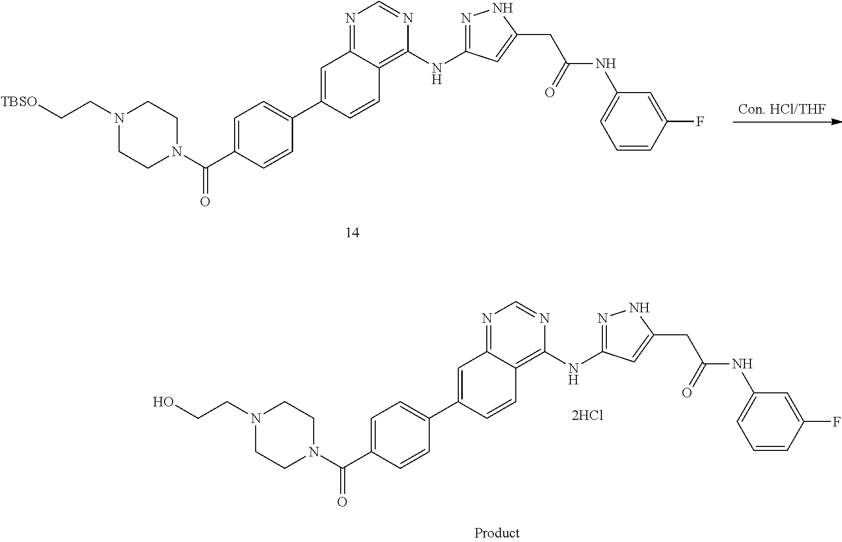

Material Resource (Reaction)

| | Material | Amount | | Equiv. | W/W | Vol/W |
|---|---|---|---|---|---|---|
| 1 | Compound 14 | 20.0 | g | 1.0 | | |
| 2 | Conc. HCl | 50 | mL | | | 2.5 |
| 3 | THF | 250 | mL | | | 12.5 |
| | Product | 16 | g | | 0.8 | |

Procedure Description:

Compound 14 (20.0 g, 0.028 mol, 1.0 eq.) was suspended in 200 mL of THF. Then the suspension was heated to 40° C. To the solution was added drop-wise with 100 mL of mixed solution of conc. HCl/THF (1:1, v/v). The suspension was stirred for additional 1 h at 40° C. HPLC analysis indicated that the compound 14 has been consumed completely. The reaction mixture was filtered to give 32 g of pale yellow solid. The solid was dried under oven (45˜50° C.) to afford 16 g of product with 99.6% HPLC purity in 85% isolated yield, as a yellow solid. LC/MS m/z 595.32 (M+1). ¹H NMR (DMSO-d₆) δ 12.34 (s (broad), 1H), 11.15 (s (broad), 1H), 10.99 (s, 1H), 9.01-9.04 (m, 2H), 8.33 (s, 1H), 8.20 (d, J=8.40 Hz, 1H), 7.93 (d, J=8.00 Hz, 2H), 7.65-7.69 (m, 3H), 7.33-7.43 (m, 2H), 6.87 (m, 1H), 6.82 (s, 1H), 4.44-4.46 (m, 2H), 3.30-3.90 (m, 9H), 3.15-3.21 (m, 3H).

### EXAMPLE 3

### N-(3-fluorophenyl)-2-(3-(7-(4-(4-methylpiperazine-1-carbonyl)phenyl)quinazolin-4-ylamino)-1H-pyrazol-5-yl)acetamide (Compound 2)

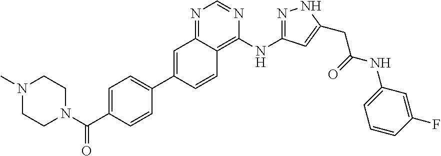

N-(3-fluorophenyl)-2-(3-(7-(4-(4-methylpiperazine-1-carbonyl)phenyl)quinazolin-4-ylamino)-1H-pyrazol-5-yl)acetamide was obtained following Example 2 and using 4-(4-methylpiperazine-1-carbonyl)phenylboronic acid. LC/MS m/z 565 (M+1). ¹H NMR (MeOD) δ 8.88 (s, 1H), 8.71 (d, J=8.5 Hz, 1H), 8.19 (dd, J=8.8, 1.7 Hz, 1H), 8.10 (d, J=1.8 Hz, 1H), 7.97 (d, J=8.2 Hz, 2H,), 7.70 (d, J=8.2 Hz, 2H), 7.57 (dt, J=11.1, 2.4 Hz, 1H), 7.26-7.35 (m, 2H), 6.86 (s, 1H), 6.82-6.85 (m, 1H), 3.88 (s, 2H), 3.6-3.20 (br s, 8H), 2.97 (s, 3H).

### EXAMPLE 4

### N-(3-fluorophenyl)-2-(3-(7-(4-(pyrrolidin-1-ylsulfonyl)phenyl)quinazolin-4-ylamino)-1H-pyrazol-5-yl)acetamide (Compound 3)

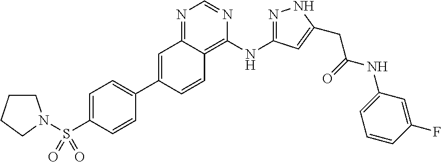

N-(3-fluorophenyl)-2-(3-(7-(4-(pyrrolidin-1-ylsulfonyl)phenyl)quinazolin-4-ylamino)-1H-pyrazol-5-yl)acetamide was obtained following Example 2 and using 4-(pyrrolidin-1-ylsulfonyl)phenylboronic acid. LC/MS m/z 572 (M+1). ¹H NMR (MeOD) δ 8.89 (s, 1H), 8.73 (d, J=8.8 Hz, 1H), 8.22 (dd, J=8.8 1.8 Hz, 1H), 8.10 (d, J=1.5 Hz, 1H), 8.05 (q, J=15.8, 8.8 Hz, 4H), 7.58 (dt, J=11, 2, 2 Hz, 1H), 7.35-7.26 (m, 2H), 6.87 (s, 1H), 6.87-6.82 (m, 1H), 3.88 (s, 2H), 3.30 (m, 4H), 1.78 (m, 4H).

### EXAMPLE 5

### N-(3-fluorophenyl)-2-(3-(7-(4-(piperazine-1-carbonyl)phenyl)quinazolin-4-ylamino)-1H-pyrazol-5-yl)acetamide (Compound 4)

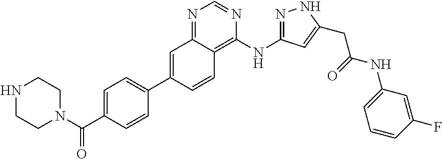

N-(3-fluorophenyl)-2-(3-(7-(4-(piperazine-1-carbonyl)phenyl)quinazolin-4-ylamino)-1H-pyrazol-5-yl)acetamide was obtained following Example 2 and using 4-(piperazine-1-carbonyl)phenylboronic acid. LC/MS m/z 551 (M+1). ¹H NMR (MeOD) δ 8.87 (s, 1H), 8.70 (d, J=9 Hz, 1H), 8.18 (dd, J=9, 2 Hz, 1H), 8.09 (d, J=2 Hz, 1H), 7.97 (d, J=8 Hz, 2H), 7.70 (d, J=8 Hz, 2H), 7.58 (dt, J=11, 2 Hz, 1H), 7.35-716 (m, 2H), 6.86-6.81 (m, 2H), 3.9 (br s, 4H), 3.87 (s, 2H), 3.32 (br m, 4H).

### EXAMPLE 6

### (S)-N-(3-fluorophenyl)-2-(3-(7-(4-(3-fluoropyrrolidin-1-ylsulfonyl)phenyl)quinazolin-4-ylamino)-1H-pyrazol-5-yl)acetamide (Compound 5)

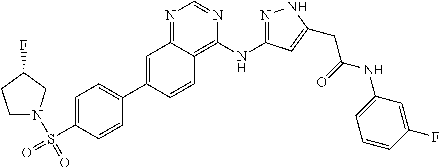

### Step 1. (S)-3-fluoro-1-(4-iodophenylsulfonyl)pyrrolidine

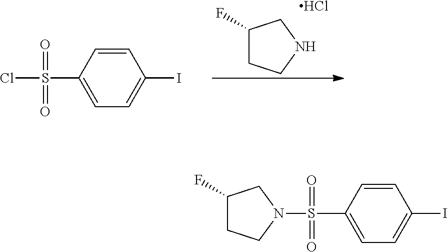

To a solution of (S)-3-fluoropyrrolidine hydrochloride (216.9 mg, 1.74 mmol) and triethylamine (416 mg, 4.13 mmol) in dichloromethane (5 mL) was added 4-iodobenzene-1-sulfonyl chloride (500 mg, 1.65 mmol) in portions at 0° C. The resultant mixture was stirred at room temperature for 3 h. The mixture was treated with water and extracted with dichloromethane. The organic layer was washed with 1 N HCl, water and brine, dried and concentrated to give (S)-3-fluoro-1-(4-iodophenylsulfonyl)pyrrolidine (465 mg, 79%). ¹H NMR (CDCl₃) δ 7.90 (d, J=8.00 Hz, 2H), 7.55 (d, J=8.40 Hz, 2H), 5.15 (m, 1H), 3.54 (m, 2H), 3.48 (m, 1H), 3.26 (m, 1H), 2.19 (m, 1H), 2.00 (m, 1H).

### Step 2. (S)-3-fluoro-1-(4-(4,4,5,5-tetramethyl-1,3,2-dioxaborolan-2-yl)phenylsulfonyl)pyrrolidine

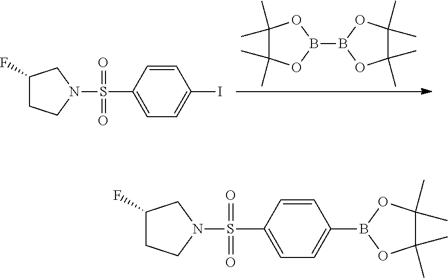

To a mixture of (S)-3-fluoro-1-(4-iodophenylsulfonyl)pyrrolidine (200 mg, 0.563 mmol), diboron pinacol ester (214 mg, 0.85 mmol), potassium acetate (193 mg, 1.97 mmol) and [1,1′-bis(diphenylphosphino)ferrocene]dichloropalladium(II) (46 mg, 0.056 mmol) in DMF was stirred at 85° C. for 3 h. The mixture was treated with water and dichloromethane. The organic layer was washed with water and brine, dried and concentrated to give the crude product. The crude product was purified by preparative TLC to give (S)-3-fluoro-1-(4-(4,4,5,5-tetramethyl-1,3,2-dioxaborolan-2-yl)phenylsulfonyl)pyrrolidine (65 mg, yield 33%). ¹H NMR (CDCl₃) δ 7.88 (d, J=8.40 Hz, 2H), 7.74 (d, J=8.40 Hz, 2H), 5.00 (m, 1H), 3.50 (m, 2H), 3.41 (m, 1H), 3.21 (m, 1H), 2.10 (m, 1H), 1.86 (m, 1H), 1.28 (s, 12H).

### Step 3. (S)-N-(3-fluorophenyl)-2-(3-(7-(4-(3-fluoropyrrolidin-1-ylsulfonyl)phenyl)quinazolin-4-ylamino)-1H-pyrazol-5-yl)acetamide

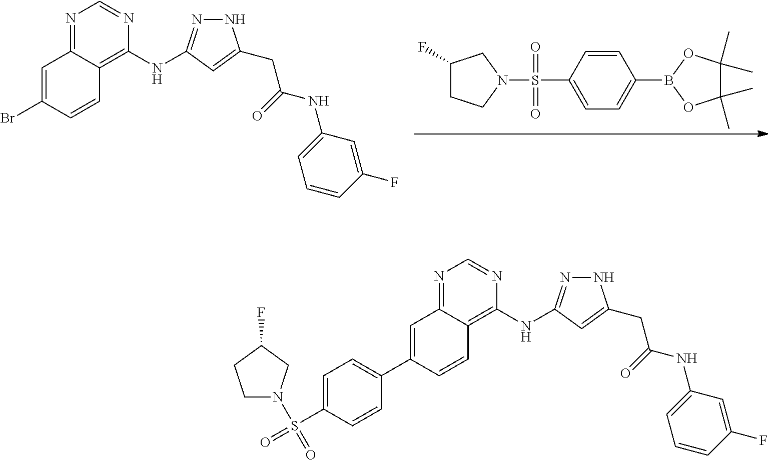

A mixture of 2-(3-(7-bromoquinazolin-4-ylamino)-1H-pyrazol-5-yl)-N-(3-fluorophenyl)acetamide (25 mg, 0.568 mmol), (S)-3-fluoro-1-(4-(4,4,5,5-tetramethyl-1,3,2-dioxaborolan-2-yl)phenylsulfonyl)pyrrolidine (30.25 mg, 0.085 mmol), 2M aqueous sodium carbonate (0.14 mL, 0.284 mmol) and [1,1′-bis(diphenylphosphino)ferrocene]dichloropalladium(II) (4.6 mg, 0.00568 mmol) in DMF was stirred at 85° C. for 3 h. After cooling, the mixture was filtered and purified by preparative HPLC to give (S)-N-(3-fluorophenyl)-2-(3-(7-(4-(3-fluoropyrrolidin-1-ylsulfonyl)phenyl)quinazolin-4-ylamino)-1H-pyrazol-5-yl)acetamide (2 mg, yield 6%). LC/MS m/z 590 (M+1). ¹H NMR (MeOD) δ 8.87 (s, 1H), 8.71 (d, J=8.80 Hz, 1H), 8.20 (d, J=10.00 Hz, 1H), 8.03 (m, 4H), 8.09 (s, 1H), 7.56 (d, J=11.60 Hz, 1H), 7.29 (m, 2H), 6.83 (m, 2H), 3.86 (s, 2H), 3.56 (m, 3H), 3.45 (m, 1H), 3.33 (m, 1H), 2.10 (m, 2H).

### EXAMPLE 7

### (R)-N-(3-fluorophenyl)-2-(3-(7-(4-(3-fluoropyrrolidin-1-ylsulfonyl)phenyl)quinazolin-4-ylamino)-1H-pyrazol-5-yl)acetamide (Compound 6)

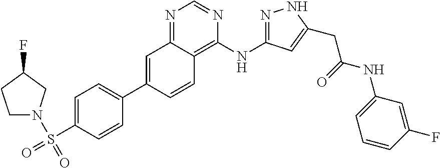

### Step 1. (R)-3-fluoro-1-(4-iodophenylsulfonyl)pyrrolidine

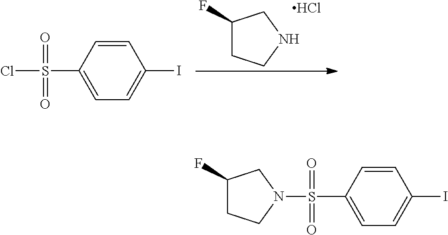

(R)-3-fluoro-1-(4-iodophenylsulfonyl)pyrrolidine was obtained following Step 1, Example 6 and using (R)-3-fluoropyrrolidine hydrochloride. ¹H NMR (CDCl₃) δ 7.97 (d, J=8.40 Hz, 2H), 7.52 (d, J=8.40 Hz, 2H), 5.18 (m, 1H), 3.55 (m, 2H), 3.46 (s, 1H), 3.23 (m, 1H), 2.15 (m, 1H), 1.92 (m, 1H).

### Step 2. (R)-3-fluoro-1-(4-(4,4,5,5-tetramethyl-1,3,2-dioxaborolan-2-yl)phenylsulfonyl)pyrrolidine

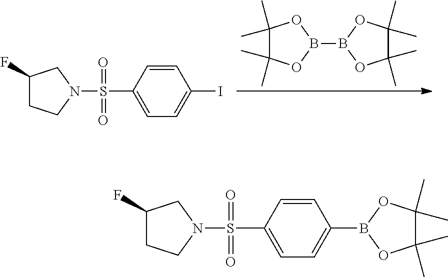

(R)-3-fluoro-1-(4-(4,4,5,5-tetramethyl-1,3,2-dioxaborolan-2-yl)phenylsulfonyl)pyrrolidine was obtained following Step 2, Example 6 and using (R)-3-fluoro-1-(4-iodophenylsulfonyl)pyrrolidine. ¹H NMR (CDCl3) δ 7.89 (d, J=8.40 Hz, 2H), 7.74 (d, J=8.40, 8.00 Hz, 2H), 5.05 (m, 1H), 3.50 (m, 2H), 3.41 (m, 1H), 3.22 (m, 1H), 2.04 (m, 1H), 1.86 (m, 1H), 1.28 (s, 12H).

### Step 3. (R)-N-(3-fluorophenyl)-2-(3-(7-(4-(3-fluoropyrrolidin-1-ylsulfonyl)phenyl)quinazolin-4-ylamino)-1H-pyrazol-5-yl)acetamide

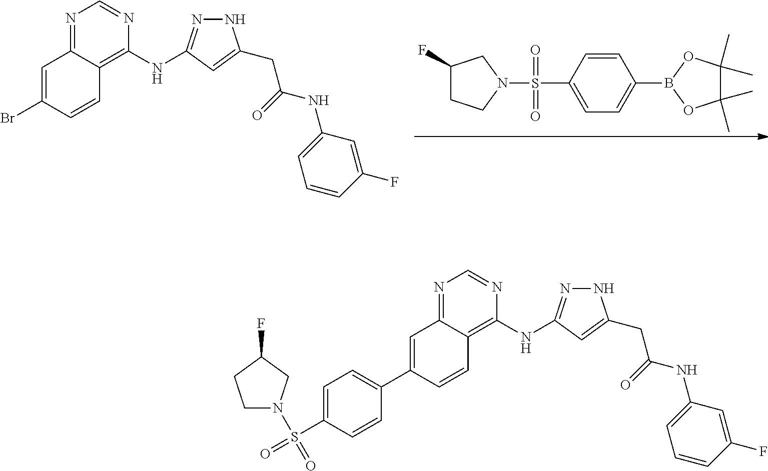

(R)-N-(3-fluorophenyl)-2-(3-(7-(4-(3-fluoropyrrolidin-1-ylsulfonyl)phenyl)quinazolin-4-ylamino)-1H-pyrazol-5-yl)acetamide was obtained following Step 3, Example 6 and using (R)-3-fluoro-1-(4-(4,4,5,5-tetramethyl-1,3,2-dioxaborolan-2-yl)phenylsulfonyl)pyrrolidine. LC/MS m/z 590 (M+1). ¹H NMR (MeOD) δ 8.84 (s, 1H), 8.68 (d, J=9.60 Hz, 1H), 8.18 (d, J=9.20 Hz, 1H), 8.08 (s, 1H), 8.05 (m, 4H), 7.54 (d, J=10.80 Hz, 1H), 7.29 (m, 2H), 6.82 (m, 2H), 3.85 (s, 2H), 3.64 (s, 1H), 3.56 (m, 3H), 3.47 (m, 1H), 2.10 (m, 2H).

### EXAMPLE 8

### N-(3-fluorophenyl)-2-(3-(7-(4-(4-methylpiperazin-1-ylsulfonyl)phenyl)quinazolin-4-ylamino)-1H-pyrazol-5-yl)acetamide (Compound 7)

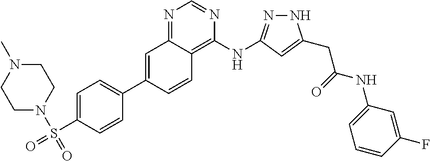

### Step 1. 1-(4-bromophenylsulfonyl)-4-methylpiperazine

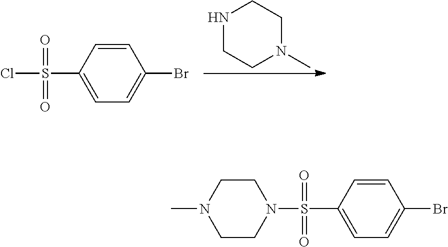

1-(4-Bromophenylsulfonyl)-4-methylpiperazine was obtained following Step 1, Example 6 and using 4-bromobenzene-1-sulfonyl chloride and 1-methylpiperazine. ¹H NMR (CDCl₃) δ 7.60 (m, 2H), 7.52 (m, 2H), 2.99 (s, 4H), 2.45 (s, 4H), 2.22 (s, 3H).

### Step 2. 1-methyl-4-(4-(4,4,5,5-tetramethyl-1,3,2-dioxaborolan-2-yl)phenylsulfonyl)piperazine

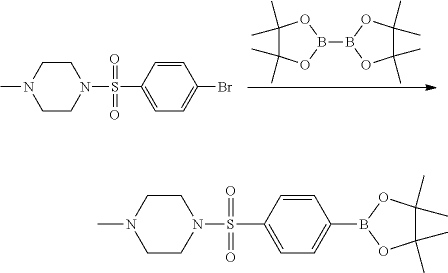

1-Methyl-4-(4-(4,4,5,5-tetramethyl-1,3,2-dioxaborolan-2-yl)phenylsulfonyl)piperazine was obtained following Step 2, Example 6 and using 1-(4-bromophenylsulfonyl)-4-methylpiperazine. ¹H NMR (CDCl₃) δ 7.99 (d, J=8.40 Hz, 2H), 7.74 (d, J=8.40 Hz, 2H), 3.89 (d, J=12.80 Hz, 2H), 3.62 (d, J=11.20 Hz, 2H), 2.96 (m, 2H), 2.87 (m, 2H), 2.82 (s, 3H), 1.35 (s, 12H).

### Step 3. N-(3-fluorophenyl)-2-(3-(7-(4-(4-methylpiperazin-1-ylsulfonyl)phenyl)quinazolin-4-ylamino)-1H-pyrazol-5-yl)acetamide

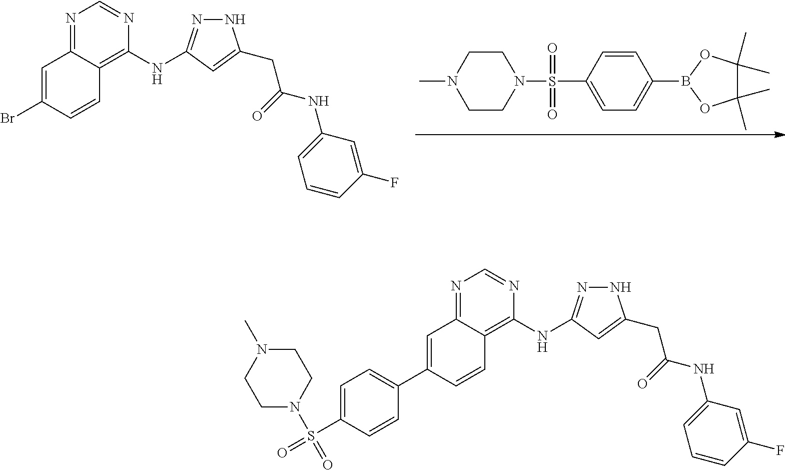

N-(3-fluorophenyl)-2-(3-(7-(4-(4-methylpiperazin-1-ylsulfonyl)phenyl)quinazolin-4-ylamino)-1H-pyrazol-5-yl)acetamide was obtained following Step 3, Example 6 and using 1-methyl-4-(4-(4,4,5,5-tetramethyl-1,3,2-dioxaborolan-2-yl)phenylsulfonyl)piperazine. LC/MS m/z 600 (M⁺). ¹H NMR (MeOD) δ 8.87 (s, 1H), 8.70 (d, J=8.40 Hz, 1H), 8.18 (dd, J=8.80, 2.00 Hz, 1H), 8.08 (m, 3H), 7.98 (d, J=8.80 Hz, 2H), 7.55 (d, J=11.20 Hz, 1H), 7.28 (m, 2H), 6.82 (m, 2H), 4.00 (m, 2H), 3.86 (s, 2H), 3.50 (m, 4H), 2.89 (s, 3H), 2.80 (m, 2H).

### EXAMPLE 9

### N-(3-fluorophenyl)-2-(3-(quinazolin-4-ylamino)-1H-pyrazol-5-yl)acetamide (Compound 8)

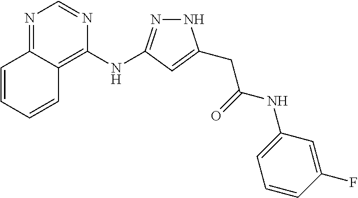

### Step 1. N-(3-fluorophenyl)-2-(3-(quinazolin-4-ylamino)-1H-pyrazol-5-yl)acetamide

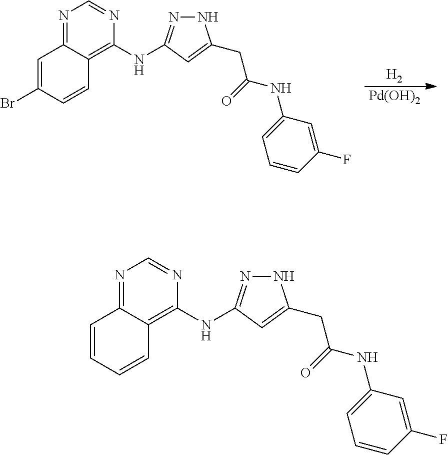

A suspension of 2-(3-(7-bromoquinazolin-4-ylamino)-1H-pyrazol-5-yl)-N-(3-fluorophenyl)acetamide (15 mg, 0.03 mmol) in MeOH (2 mL) was stirred with Pd(OH)₂ (5 mg) under H₂ for 1.5 hrs. The catalyst was filtered off and the filtrate was concentrated to give the crude product, which was purified by preparative HPLC to give N-(3-fluorophenyl)-2-(3-(quinazolin-4-ylamino)-1H-pyrazol-5-yl)acetamide (2.21 mg, yield 14%). LC/MS m/z 363 (M+1). ¹H NMR (MeOD) δ 8.76 (s, 1H), 8.52 (d, J=8.00 Hz, 2H), 8.02 (t, J=7.60 Hz, 1H), 7.79 (t, J=4.00 Hz, 2H), 7.49 (d, J=10.80 Hz, 1H), 7.23 (m, 2H), 6.76 (d, J=8.00 Hz, 2H), 3.79 (s, 2H).

### EXAMPLE 10

### 2-(3-(7-(4-(4-(Cyclopropylmethyl)piperazine-1-carbonyl)phenyl)quinazolin-4-ylamino)-1H-pyrazol-5-yl)-N-(3-fluorophenyl)acetamide (Compound 9)

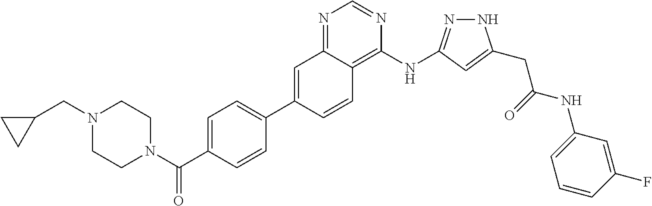

### Step 1. methyl 4-(4-(5-(2-(3-fluorophenylamino)-2-oxoethyl)-1H-pyrazol-3-ylamino)quinazolin-7-yl)benzoate

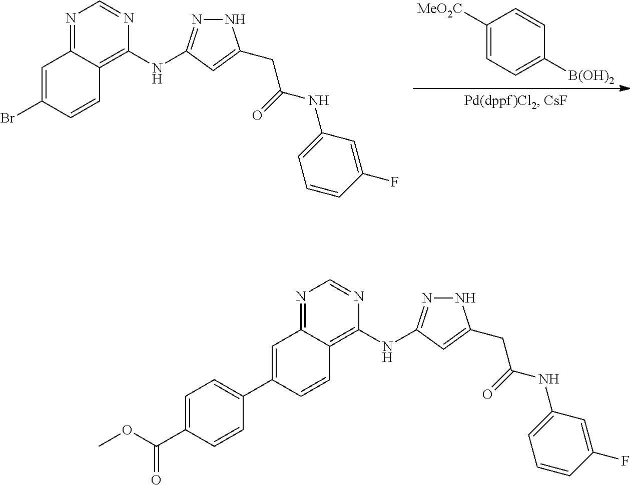

A mixture of 2-[5-(7-bromo-quinazolin-4-ylamino)-2H-pyrazol-3-yl]-N-(3-fluorophenyl)acetamide (2 g, 4.6 mmol), 4-(methoxycarbonyl)phenylboronic acid (1.72 g, 9.6 mmol), CsF (3.44 g, 22.6 mmol), 18-crown-6 (0.6 g, 2.27 mmol) and Pd(dppf)Cl₂ (0.6 g, 0.79 mmol) in 1,4-dioxane (200 mL) and water (20 mL) was heated at 90° C. for overnight under Ar. The mixture was cooled to room temperature and treated with brine (10 mL) and i-PrOH/EtOAc (v:v, 30 mL). The separated aqueous layer was extracted with i-PrOH/EA (v:v, 30 mL) for another two times. The combined organic layers were washed with aqueous K₂CO₃ solution (10%, 30 mL) and brine (10 mL); dried and concentrated to give the crude product, which was purified by preparative HPLC to give methyl 4-(4-(5-(2-(3-fluorophenylamino)-2-oxoethyl)-1H-pyrazol-3-ylamino)quinazolin-7-yl)benzoate (1.3 g, 58%). ¹H NMR (MeOD): 3.87 (s, 2H), 3.96 (s, 3H), 6.85 (m, 2H), 7.28 (m, 2H), 7.58 (d, 1H), 7.96 (d, 2H), 8.09 (s, 1H), 8.20 (d, 3H), 8.71 (d, 1H), 8.88 (s, 1H).

### Step 2. sodium 4-(4-(5-(2-(3-fluorophenylamino)-2-oxoethyl)-1H-pyrazol-3-ylamino)quinazolin-7-yl)benzoate

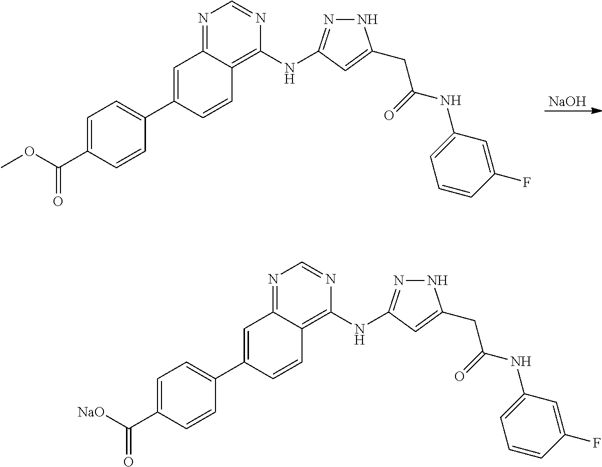

A mixture of methyl 4-(4-(5-(2-(3-fluorophenylamino)-2-oxoethyl)-1H-pyrazol-3-ylamino)quinazolin-7-yl)benzoate (700 mg, 1.6 mmol) and NaOH (64 mg, 1.6 mmol) in MeOH (5 mL) was refluxed for overnight. The mixture was concentrated to give sodium 4-(4-(5-(2-(3-fluorophenylamino)-2-oxoethyl)-1H-pyrazol-3-ylamino)quinazolin-7-yl)benzoate (720 mg, crude).

### Step 3. 2-(3-(7-(4-(4-(cyclopropylmethyl)piperazine-1-carbonyl)phenyl)quinazolin-4-ylamino)-1H-pyrazol-5-yl)-N-(3-fluorophenyl)acetamide

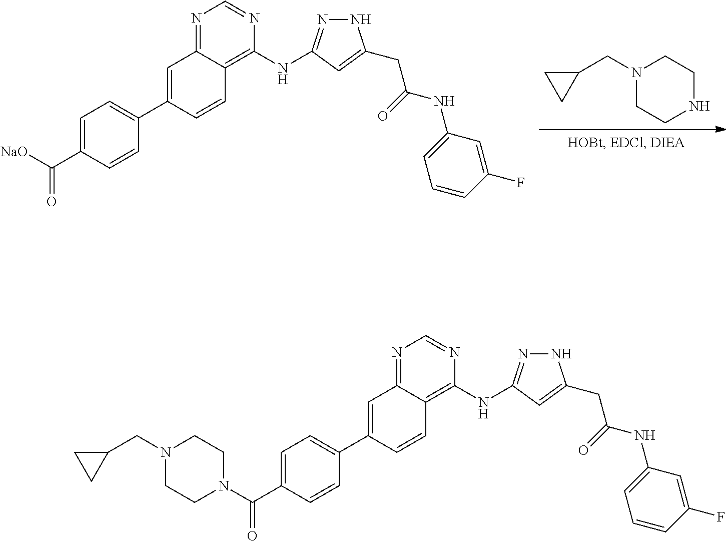

A mixture of sodium 4-(4-(5-(2-(3-fluorophenylamino)-2-oxoethyl)-1H-pyrazol-3-ylamino)quinazolin-7-yl)benzoate (150 mg, 298 μmol), EDCI (117 mg, 591 μmol), HOBt (80 mg, 593 μmol), 1-(cyclopropylmethyl)piperazine (50 mg, 357 μmol) and DIEA (1.6 g, 1186 μmol) in DMF (4 mL) was stirred at room temperature for overnight. The mixture was purified by preparative HPLC to give 2-(3-(7-(4-(4-(cyclopropylmethyl)piperazine-1-carbonyl)phenyl)quinazolin-4-ylamino)-1H-pyrazol-5-yl)-N-(3-fluorophenyl)acetamide (28.29 mg, 16%). ¹H NMR (MeOD): 0.46 (m, 2H), 0.79 (m, 2H), 1.15 (m, 1H), 2.68-3.20 (m, 6H), 3.35-3.80 (m, 4H), 3.88 (s, 2H), 6.85 (m, 2H), 7.30 (m, 2H), 7.57 (d, 1H), 7.71 (d, 2H), 7.98 (d, 2H), 8.08 (s, 1H), 8.18 (d, 1H), 8.71 (d, 1H), 8.88 (s, 1H).

### EXAMPLE 11

### 2-(3-(7-(4-(4-Cyclopropylpiperazine-1-carbonyl)phenyl)quinazolin-4-ylamino)-1H-pyrazol-5-yl)-N-(3-fluorophenyl)acetamide (Compound 10)

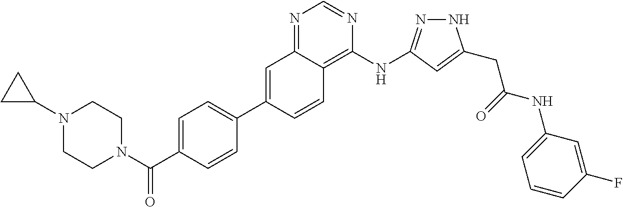

2-(3-(7-(4-(4-Cyclopropylpiperazine-1-carbonyl)phenyl)quinazolin-4-ylamino)-1H-pyrazol-5-yl)-N-(3-fluorophenyl)acetamide was obtained (22% yield) following Step 3, Example 10 and using 1-cyclopropylpiperazine hydrogenchloride. ¹H NMR (MeOD): 0.98 (m, 4H), 2.84 (m, 1H), 3.46 (m, 4H), 3.86 (m, 6H), 6.83 (m, 2H), 7.30 (m, 2H), 7.57 (d, 1H), 7.70 (d, 2H), 7.97 (d, 2H), 8.08 (s, 1H), 8.19 (d, 1H), 8.70 (d, 1H), 8.87 (s, 1H).

### Preparation of 1-cyclopropylpiperazine hydrogenchloride

### Step 1. 4-cyclopropyl-piperazine-1-carboxylic acid tert-butyl ester

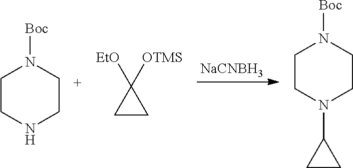

A mixture of piperazine-1-carboxylic acid tert-butyl ester (2.5 g, 13.4 mmol), (1-ethoxy-cyclo propoxy)-trimethyl-silane (5 g, 28.7 mmol), NaBH₃CN (7.6 g, 120.9 mmol) and acetic acid (1.3 mL) in THF (30 mL) and MeOH (30 mL) was heated at 60° C. for 5 hrs. The mixture was treated with water and aqueous 1N NaOH solution. The mixture was concentrated and the remained aqueous layer was extracted with DCM (50 mL*3). The combined organic layer was dried and concentrated to give the crude product, which was purified by silica column chromatography to give tert-butyl 4-cyclopropylpiperazine-1-carboxylate (1.5 g, crude).

### Step 2. 1-cyclopropylpiperazine hydrochloride

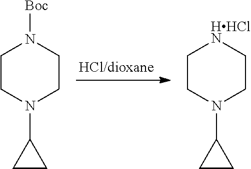

A mixture of tert-butyl 4-cyclopropylpiperazine-1-carboxylate (600 mg, crude) dissolved in the solution of 4 N hydrogen chloride in dioxane (10 mL) was stirred at room temperature for overnight. The reaction mixture was evaporated under reduced pressure to give 1-cyclopropylpiperazine hydrochloride (460 mg, crude).

### EXAMPLE 12

### 2-(3-(7-(4-((3S,5R)-3,5-dimethylpiperazine-1-carbonyl)phenyl)quinazolin-4-ylamino)-1H-pyrazol-5-yl)-N-(3-fluorophenyl)acetamide (Compound 11)

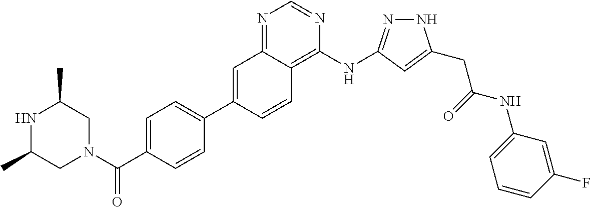

2-(3-(7-(4-((3S,5R)-3,5-dimethylpiperazine-1-carbonyl)phenyl)quinazolin-4-ylamino)-1H-pyrazol-5-yl)-N-(3-fluorophenyl)acetamide was obtained (20% yield) following step 3, Example 10 and using (2R,6S)-2,6-dimethylpiperazine. ¹H NMR (MeOD): 1.34 (m, 6H), 3.35-3.58 (m, 6H), 3.87 (s, 2H), 6.84 (m, 2H), 7.30 (m, 2H), 7.58 (d, 1H), 7.70 (d, 2H), 7.98 (d, 2H), 8.09 (s, 1H), 8.17 (d, 1H), 8.70 (d, 1H), 8.87 (s, 1H).

### EXAMPLE 13

### 2-(3-(7-(4-(4-(2-Fluoroethyl)piperazine-1-carbonyl)phenyl)quinazolin-4-ylamino)-1H-pyrazol-5-yl)-N-(3-fluorophenyl)acetamide (Compound 12)

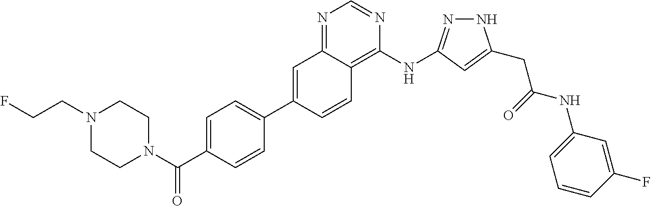

2-(3-(7-(4-(4-(2-fluoroethyl)piperazine-1-carbonyl)phenyl)quinazolin-4-ylamino)-1H-pyrazol-5-yl)-N-(3-fluorophenyl)acetamide was obtained following step 3, Example 10 and using 4-(2-Fluoro-ethyl)-piperidine. ¹H NMR (D₂O): 8.61 (s, 1H), 8.27-8.25 (d, 1H), 7.95 (d, 1H), 7.82 (s, 1H), 7.74-7.72 (d, 2H), 7.46-7.44 (d, 2H), 7.25-7.15 (m, 2H), 7.05-7.03 (m, 1H), 7.25-7.15 (m, 2H), 6.81-6.80 (m, 1H), 6.45 (s, 1H), 4.81-4.71 (m, 4H), 4.0-3.2 (m, 10H).

### EXAMPLE 14

### 2-(4-(4-(4-(5-(2-(3-Fluorophenylamino)-2-oxoethyl)-1H-pyrazol-3-ylamino)quinazolin-7-yl)benzoyl)piperazin-1-yl)ethyl dihydrogen phosphate (Compound 13)

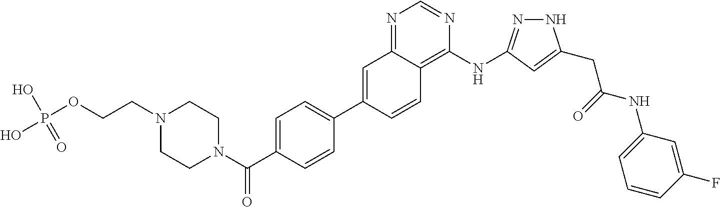

### Step 1. 2-(4-(4-(4-(5-(2-(3-fluorophenylamino)-2-oxoethyl)-1H-pyrazol-3-ylamino)quinazolin-7-yl)benzoyl)piperazin-1-yl)ethyl dihydrogen phosphate

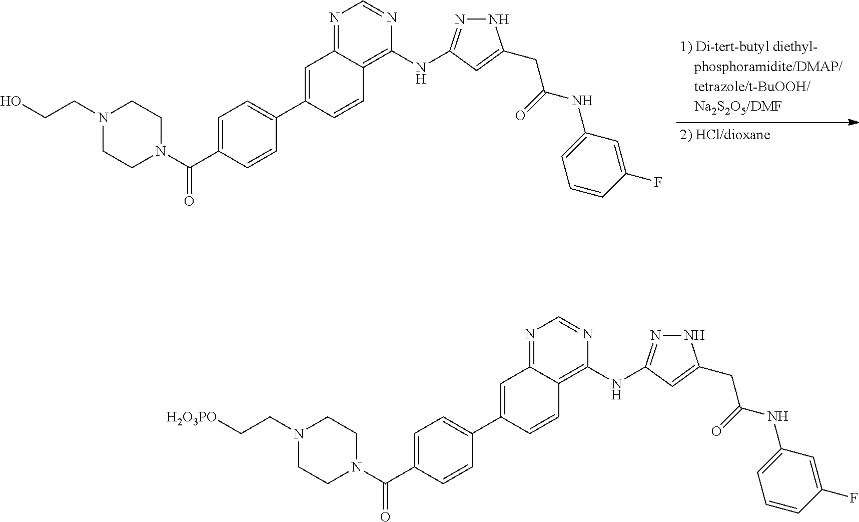

Di-tert-butyldiethylphosphoramidite (2.1 g, 8.4 mmol) was slowly added to a solution of N-(3-fluorophenyl)-2-(3-(7-(4-(4-(2-hydroxyethyl)piperazine-1-carbonyl)phenyl)quinazolin-4-ylamino)-1H-pyrazol-5-yl)acetamide (500 mg, 0.84 mmol) in DMF (5 mL) in the presence of tetrazole (588 mg, 8.4 mmol) and DMAP (108 mg, 0.84 mmol) at ambient temperature under N₂. The mixture was stirred at ambient temperature for overnight and was then cooled to 0° C. ^{t}BuOOH (1.26 g, 60%, 8.4 mmol) was slowly added and the resulting mixture was stirred at ambient temperature for 2 h. The mixture was cooled to 0° C., and a solution of sodium metabisulphite in water (10%, 2 mL) was added. The mixture was allowed to be warmed to ambient temperature. After stirring for 0.5 h, the mixture was treated with ethyl acetate and water. The layers were separated and the aqueous solution was extracted with ethyl acetate. The combined organic layer was washed with water and brine, dried and concentrated. The residue was dissolved in dichloromethane (20 mL), and a solution of hydrogen chloride in dioxane (4 M, 20 mL) was added. The mixture was stirred at room temperature for 15 h and then heated to 40-45° C. for 2 h. The mixture was filtered, and the solid was purified by preparative HPLC to give 2-(4-(4-(4-(5-(2-(3-fluorophenyl amino)-2-oxoethyl)-1H-pyrazol-3-ylamino)quinazolin-7-yl)benzoyl)piperazin-1-yl)ethyldihydrogen phosphate (100.22 mg, yield 18%): ¹H NMR (MeOD): 3.35 (m, 6H), 3.72 (m, 1H), 3.78 (s, 2H), 3.90 (m, 3H), 4.14 (m, 2H), 6.77 (m, 2H), 7.21 (m, 2H), 7.48 (m, 1H), 7.61 (d, 2H), 7.87 (d, 2H), 8.00 (d, 1H), 8.10 (dd, 1H), 8.62 (d, 1H), 8.79 (s, 1H).

### EXAMPLE 15

### N-(3,5-Difluorophenyl)-2-(3-(7-(4-(4-(2-hydroxyethyl)piperazine-1-carbonyl)phenyl)quinazolin-4-ylamino)-1H-pyrazol-5-yl)acetamide (Compound 14)

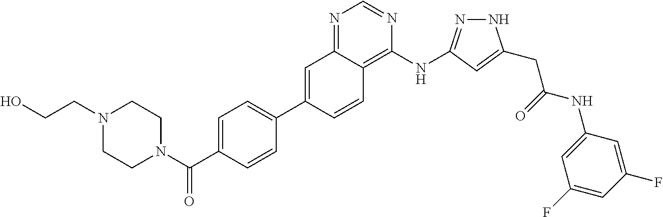

N-(3,5-Difluoro phenyl)-2-(3-(7-(4-(4-(2-hydroxyethyl)piperazine-1-carbonyl)phenyl)quinazolin-4-ylamino)-1H-pyrazol-5-yl)acetamide was obtained following the similar procedure of Example 2 and by substitution of 3-fluorobenzenamine with 3,5-difluorobenzenamine in step 4 of Example 1. ¹H NMR (MeOD): 3.35 (t, 4H), 3.36-3.80 (m, 6H), 3.88 (s, 2H), 3.91 (t, 4H), 6.68 (m, 1H), 6.86 (s, 1H), 7.28 (d, 2H), 7.71 (d, 2H), 7.98 (d, 2H), 8.10 (s, 1H), 8.18 (d, 1H), 8.71 (d, 1H), 8.89 (s, 1H).

### EXAMPLE 16

### N-(2,3-Difluorophenyl)-2-(3-(7-(4-(4-(2-hydroxyethyl)piperazine-1-carbonyl)phenyl)quinazolin-4-ylamino)-1H-pyrazol-5-yl)acetamide (Compound 15)

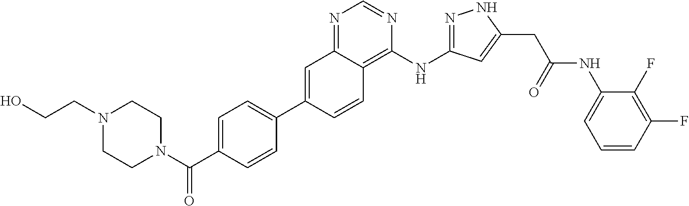

N-(2,3-difluoro phenyl)-2-(3-(7-(4-(4-(2-hydroxyethyl)piperazine-1-carbonyl)phenyl)quinazolin-4-ylamino)-1H-pyrazol-5-yl)acetamide was obtained following the similar procedure of Example 2 and by substitution of 3-fluorobenzenamine with 2,3-difluorobenzenamine in step 4 of Example 1. ¹H NMR (MeOD): 3.35 (m, 4H), 3.47-3.89 (m, 6H), 3.91 (t, 2H), 3.95 (s, 1H), 6.87 (s, 1H), 7.06-7.14 (m, 2H), 7.70 (m, 3H), 7.98 (d, 2H), 8.10 (s, 1H), 8.18 (d, 2H), 8.70 (d, 1H), 8.88 (s, 1H).

### EXAMPLE 17

### The Disclosed Compounds are Active in an Enzyme Activity Assay for Aurora Kinases

Kinase activity of Aurora kinases was measured by phosphocellulose filter binding assay. The full-length human Aurora A carrying His₆ tag at the N-terminus (Invitrogen, Carlsbad, Calif.) and the full-length human Aurora B (N-terminal GST fusion) co-expressed and co-purified with the His₆-INCENP INBOX, aminoacids 803-918 (Carna BioSciences, Japan) were used as the enzyme in the assays. Tested compounds were diluted in DMSO and pre-incubated with the enzyme and ATP/Mg²⁺ mixture for at least 10 minutes. The reaction was started by addition of the Kemptide peptide substrate (LRRASLG) and allowed to proceed for 45 min at room temperature. Each reaction in a final volume of 40 μl contained 50 μM Kemptide, 10 μM [γ-³³P]ATP (1 Ci/mmol) and either 11 nM Aurora A or 1.5 nM Aurora B/INCENP complex in the buffer containing 10 mM MgCl₂, 5% DMSO, 20 mM MOPS, pH 7.2, 25 mM β-glycerol phosphate, 5 mM EGTA, 1 mM Na₃VO₄ and either 0.1 mg/ml of BSA and 1 mM DTT for Aurora A or 0.01% Triton-X100 and 2 mM DTT for Aurora B. The reaction was stopped with 25 μl of 5% H₃PO₄, and 45 μl of the mixture was transferred into a MultiScreen-PH filterplate (Millipore) pre-washed with 0.85% H₃PO₄. The filterplate was washed 4 times with 200 μl of 0.85% H₃PO₄ on a vacuum manifold. Scintillation fluid (50 μl of Ultima Gold™ LLT, PerkinElmer, Waltham, Mass.) was added to each well, and after 1-hr incubation at room temperature the plates were counted on MicroBeta (PerkinElmer, Waltham, Mass.) plate reader. Incubation with 5% DMSO in the absence of inhibitors was used as a non-inhibited control, and the reaction mixture lacking Kemptide was used as a fully inhibited control. Percent inhibition values were calculated based on the counts of the positive and negative controls, and these values were regressed against inhibitor concentration and fit into a four parameter model. From this fit the IC₅₀ values (the inhibitor concentration at which 50% inhibition occurs) were derived as the inflection point on the semi-logarithm inhibition curve. Aurora kinase A and B IC₅₀s for a number of the disclosed compounds are shown below in Table 1. For reference purposes, the IC₅₀s of AZD1152HQPA and VX-680, two Aurora kinase inhibitors known in the prior art and in clinical development, are also included in Table 1. The structures of AZD1152HQPA and VX-680 are shown below:

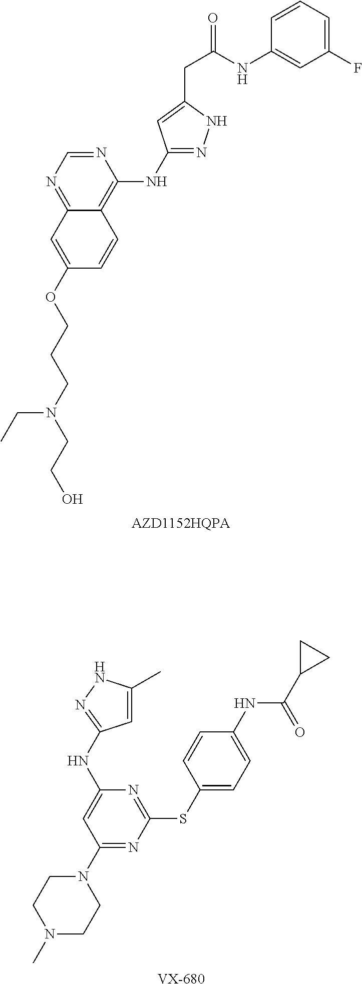

TABLE 1
Enzyme assay data:

| | | |
|---|---|---|
| Compound | Aur-A | Aur-B |

| Taxol | NA | | NA | |
|---|---|---|---|---|
| Compound 1 | 1.93 | nM | 0.48 | nM |
| Compound 2 | 5.07 | nM | 0.86 | nM |
| Compound 3 | 6.04 | nM | 0.47 | nM |
| Compound 4 | 3.77 | nM | 0.71 | nM |
| Compound 5 | 7.1 | nM | 0.56 | nM |
| Compound 6 | 6.29 | nM | 0.63 | nM |
| Compound 7 | 13.94 | nM | 0.93 | nM |
| Compound 8 | 2478.6 | nM | 14.95 | nM |
| Compound 9 | 9.51 | nM | 1.88 | nM |
| Compound 10 | 4.62 | nM | 1.23 | nM |
| Compound 11 | 6.42 | nM | 1.37 | nM |
| Compound 12 | 2.86 | nM | 0.74 | nM |
| Compound 13 | 1.91 | nM | 0.34 | nM |
| Compound 14 | 2.29 | nM | 0.83 | nM |
| Compound 15 | 1.89 | nM | 0.63 | nM |
| AZD1152HQPA | 300 | nM | 1 | nM |
| VX-680 | 2 | nM | 10 | nM |

### EXAMPLE 18

### The Disclosed Compounds Have Activity Against Cancer Cells in Vivo

The CellTiter-Glo® Luminescent Cell Viability Assay is a homogeneous method of determining the number of viable cells in culture based on quantitation of the ATP present, which signals the presence of metabolically active cells. The homogeneous assay procedure involves addition of a single reagent directly to cells cultured in serum-supplemented medium. The homogeneous format results in cell lysis and generation of a luminescent signal proportional to the amount of ATP present. The CellTiter-Glo® Assay relies on the properties of a proprietary thermostable luciferase (Ultra-Glo. Recombinant Luciferase), which generates a stable glow-type Luminescent signal. The half-life of the luminescent signal resulting from this reaction is greater than five hours. This extended half-life eliminates the need for reagent injectors and provides flexibility for continuous or batch-mode processing of multiple plates. The unique homogeneous format reduces pipetting errors that may be introduced during the multiple steps required by other ATP-measurement methods.

### Protocol for the Cell Viability Assay:

96 Well cell culture plates were seeded with 10³ mammalian cells (Colo205, HL-60, SW620, HCT116, MCF-7 and MV-411) in 100 μl culture medium. The cells were incubated overnight prior to initiating the assay. Control wells were prepared on a plate containing medium only to obtain a value for background luminescence.

Compounds to be tested were added to experimental wells and incubated for 72 hrs (Colo205, HL-60, MV-411), 168 hrs (HCT116, SW620) or 240 hrs (MCF-7). Wells were also set up to as vehicle controls with the addition of 0.1% DMSO. At the end of the incubation period, the plates were equilibrated to room temperature for approximately 30 minutes.

Lyophilized CellTiter-Glo® Substrate and the CellTiter-Glo® Buffer were warmed to room temperature. The CellTiter-Glo® Buffer was mixed with the CellTiter-Glo® Substrate to reconstitute the lyophilized enzyme/substrate mixture. This forms the CellTiter-Glo® Reagent. The contents were mixed by gently vortexing, swirling or by inverting the contents to obtain a homogeneous solution. 100 μl of CellTiter-Glo® Reagent was added to the cell culture medium present in each well. The contents were mixed for 2 minutes on an orbital shaker to induce cell lysis. The plates were allowed to incubate at room temperature for 10 minutes to stabilize luminescent signal. The contents of the 96 well cell culture plate were then transferred to a white 96 well Optiplate (Perkin Elmer). The luminescence was recorded on Wallac 1420 VICTOR² V Multilabel Platereader (An integration time of 0.25-1 second per well).

### Preparation of Test Compounds:

10 mM DMSO stock Compounds were serially diluted threefold in DMSO. Subsequent dilutions made into cell culture media prior to addition to cells to maintain the DMSO concentration at or below 0.1% to avoid cell toxicity.

The IC₅₀s of compounds of the invention and AZD1152HQPA and VX-680, two Aurora kinase inhibitors known in the prior art and in clinical development, are also included in Table 2. The IC₅₀s in Table 2 are the concentration at which 50% growth inhibition relative to control occurs.

TABLE 2
Cell assay data: Results are expressed as IC₅₀ (nM).

| Compound | Colo205 | HL-60 | SW620 | HCT116 | MCF-7 | MV-411 |
|---|---|---|---|---|---|---|
| Taxol | 2.54 nM | 2.16 nM | 4.91 nM | 2.04 nM | 0.58 nM | 6.2 nM |
| Compound 1 | 1.82 nM | 1.32 nM | 12.01 nM | 5.32 nM | 16.91 nM | 5.75 nM |
| Compound 2 | 3.98 nM | 1.8 nM | 6.3 nM | 3.65 nM | 8.46 nM | 5.2 nM |
| Compound 3 | 2.26 nM | 1.08 nM | 2.15 nM | 4.58 nM | 5.62 nM | 5.9 nM |
| Compound 4 | 2.11 nM | 1.32 nM | 7.81 nM | 5.66 nM | 7.7 nM | 6.05 nM |
| Compound 5 | 3.86 nM | 1.21 nM | 2.17 nM | 1.9 nM | 11.42 nM | 6.4 nM |
| Compound 6 | 3.9 nM | 2.26 nM | 2.4 nM | 1.9 nM | 7.8 nM | 5.79 nM |
| Compound 9 | 9.88 nM | | | 4.38 nM | | |
| Compound 10 | 5.35 nM | | | 1.57 nM | | |
| Compound 11 | 2.90 nM | | | 1.62 nM | | |
| Compound 12 | 7.82 nM | | | 3.98 nM | | |
| Compound 13 | 9.39 nM | | | 6.43 nM | | |
| Compound 14 | 8.11 nM | | | 5.75 nM | | |
| Compound 15 | 3.07 nM | | | 1.71 nM | | |
| AZD1152HQPA | 22.98 nM | 7.16 nM | 20.91 nM | 14.35 nM | 60.74 nM | 16.5 nM |
| VX-680 | 30.22 nM | 13.77 nM | 21.81 nM | 17.97 nM | 31 nM | 79 nM |

Colo205: Colorectal cancer
HL-60: Leukemia
SW620: Colorectal cancer
HCT116: Colorectal cancer
MCF-7: Breast Cancer
MV-411: Leukemia

While this invention has been particularly shown and described with references to example embodiments thereof, it will be understood by those skilled in the art that various changes in form and details may be made therein without departing from the scope of the invention encompassed by the appended claims.

1. A compound represented by the following structural formula: 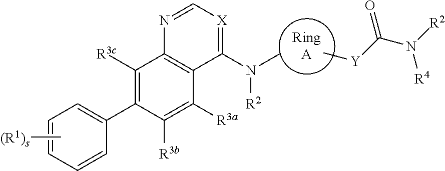
or a pharmaceutically acceptable salt thereof; wherein:
X is CR^{3d} or N;
Y is a (C₁-C₃)alkylene or NR²;
each R¹ is independently halo, OR¹¹, S(O)ₚR¹¹, CN, NO₂, CO₂R¹¹, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹¹R¹², CONR¹¹R¹², OC(O)NR¹¹R¹², NR¹¹C(O)NR¹¹R¹², CSNR¹¹R¹², OC(S)NR¹¹R¹², NR¹¹C(S)NR¹¹R¹², SO₂NR¹¹R¹², NR¹¹SO₂NR¹¹R¹², NR¹¹C(O)R¹², OC(O)R¹², NR¹¹C(S)R¹², NR¹¹C(O)OR¹², NR¹¹C(S)OR¹², or NR¹¹SO₂R¹²;
each R² is independently H or (C₁-C₃)alkyl;
R^{3a} is H, halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴;
the (C₆-C₁₀)aryl and (5 to 10 membered)heteroaryl represented by R¹ and R^{3a} are optionally and independently substituted with 1 to 4 substituents selected from halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴;
the (C₃-C₈)cycloalkyl and the (3 to 9 membered)heterocyclyl represented by R¹ and R^{3a} are optionally and independently substituted with oxo, halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN;
R^{3b} and R^{3d} are each independently H, halo, CN, NO₂, OH, (C₁-C₆)alkyl, halo(C₁-C₆)alkyl, (C₁-C₆)alkoxy, amino, (C₁-C₆)alkylamino, di(C₁-C₆)alkylamino, or halo(C₁-C₆)alkoxy;
R^{3c} is H or F;
R⁴ is (C₆-C₁₀)aryl, (5 to 10 membered)heteroaryl, (C₆-C₁₀)aryl(C₁-C₄)alkyl, (5 to 10 membered)heteroaryl(C₁-C₄)alkyl, (C₁-C₆)alkyl, (C₃-C₈)cycloalkyl, or (3 to 9 membered)heterocyclyl, each optionally and independently substituted with 1 to 4 substituents selected from the group consisting of halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (3 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, OC(O)R¹⁴, NR¹³C(S)R¹⁴, NR¹³C(O)OR¹⁴, NR¹³C(S)OR¹⁴, or NR¹³SO₂R¹⁴; or R² and R⁴, taken together with the nitrogen to which they are attached form a 3 to 9 membered heterocycle, optionally containing 1 additional ring heteroatom selected from oxygen, nitrogen and sulfur, and optionally substituted with halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN; and
each R¹¹ and each R¹² are independently H, (C₁-C₄)alkyl, (C₃-C₈)cycloalkyl, (C₃-C₈)cycloalkyl(C₁-C₄)alkyl, halo(C₁-C₄)alkyl, (C₁-C₄)alkoxy(C₁-C₄)alkyl, or hydroxy(C₁-C₄)alkyl, wherein the (C₃-C₈)cycloalkyl and (C₃-C₈)cycloalkyl(C₁-C₄)alkyl are optionally and independently substituted with one or two groups selected from oxo, (C₁-C₂)alkyl, hydroxy, (C₁-C₂)alkoxy, amino, (C₁-C₆)alkylamino, di(C₁-C₆)alkylamino or halo; or
R¹¹ and R¹², taken together with the nitrogen atom to which they are attached, form a 3 to 9 membered nitrogen-containing heterocycle, optionally containing 1 additional ring heteroatom selected from oxygen, nitrogen and sulfur, and optionally substituted with oxo, halo, (C₁-C₄)alkyl, (C₃-C₈)cycloalkyl, Spiro (C₃-C₈)cycloalkyl, (C₃-C₈)cycloalkyl(C₁-C₄)alkyl, (C₁-C₄)hydroxyalkyl, —(CH₂)_{q}-—OR, (C₁-C₄)alkoxyalkyl, (C₁-C₄)alkoxy, (C₃-C₈)cycloalkoxy, (C₃-C₈)cycloalkyl(C₁-C₄)alkoxy, (C₁-C₄)thio alkyl, (C₃-C₈)cycloalkylthio, (C₃-C₈)cycloalkyl(C₁-C₄)thioalkyl, halo(C₁-C₄)alkyl, halo(C₁-C₄)alkoxy, amino, (C₁-C₄)alkylamino, (C₃-C₈)cycloalkylamino, (C₃-C₈)cycloalkyl(C₁-C₄)alkylamino, [(C₃-C₈)cycloalkyl(C₁-C₄)alkyl][(C₁-C₄)alkyl]amino, [(C₃-C₈)cycloalkyl(C₁-C₄)alkyl][(C₃-C₈)cycloalkyl]amino, di(C₁-C₄)alkylamino, di(C₃-C₈)cycloalkylamino, di[(C₃-C₈)cycloalkyl(C₁-C₄)alkyl]amino, aminocarbonyl, (C₁-C₄)alkylaminocarbonyl, (C₃-C₈)cycloalkylaminocarbonyl, (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl, di[(C₃-C₈)cycloalkyl(C₁-C₄)alkyl]carbonyl, (C₁-C₄)alkylcarbonyl, (C₃-C₈)cycloalkylcarbonyl, (C₃-C₈)cycloalkyl(C₁-C₄)alkylcarbonyl, (C₁-C₄)alkoxycarbonyl, (C₃-C₈)cycloalkoxycarbonyl, (C₃-C₈)cycloalkyl(C₁-C₄)alkoxycarbonyl, S(O)ₚ(C₁-C₄)alkyl, NO₂, OH, CN or 3 to 9 membered nitrogen-containing heterocyclyl optionally containing 1 additional ring heteroatom selected from oxygen, nitrogen or sulfur; and
each R¹³ and each R¹⁴ are independently H, (C₁-C₄)alkyl, halo(C₁-C₄)alkyl or hydroxy(C₁-C₄)alkyl
s is an integer from 0 to 3;
q is an integer from 1 to 4;
p is an integer from 0 to 2;
R is P(O)(OR′)₂, P(O)(OR′)₃, S(O)(OR′), S(O)(OR′)₂, C(O)R′, C(O)N(R′)₂, P(S)(OR′)₂, P(S)(OR′)₃, C(S)R′, or C(O)N(R′)₂;
R′ is H, (C₁-C₄)alkyl, (C₁-C₄)hydroxyalkyl, (C₁-C₄)alkoxyalkyl, or phenyl optionally substituted with halo, (C₁-C₄)alkyl, (C₁-C₄)alkoxy, amino, (C₁-C₆)alkylamino, di(C₁-C₆)alkylamino, halo(C₁-C₄)alkyl, halo(C₁-C₄)alkoxy, OH, NO₂ or CN; and
Ring A is a (C₆-C₁₀)aryl or (5-10 membered)heteroaryl, optionally substituted with halo, (C₁-C₄)alkyl, (C₁-C₄)alkoxy, amino, (C₁-C₆)alkylamino, di(C₁-C₆)alkylamino, halo(C₁-C₄)alkyl, halo(C₁-C₄)alkoxy, OH, NO₂ or CN.

2. The compound according to claim 1, wherein the compound is represented by a structural formula selected from: 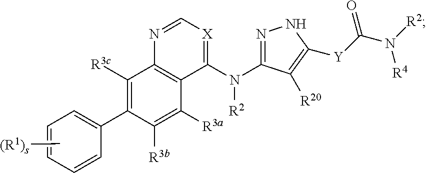
and
or a pharmaceutically acceptable salt thereof, wherein R²⁰ is H, halo, (C₁-C₄)alkyl, (C₁-C₄)alkoxy, halo(C₁-C₄)alkyl, halo(C₁-C₄)alkoxy, NH₂, (C₁-C₄)alkylamine, di(C₁-C₄)alkylamine, OH, NO₂ or CN.

3. The compound according to claim 2, wherein the compound is represented by the following structural formula: 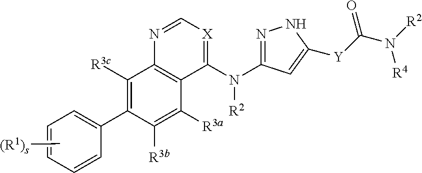
or a pharmaceutically acceptable salt thereof.

4. The compound according to claim 3, wherein the compound is represented by the following structural formula: 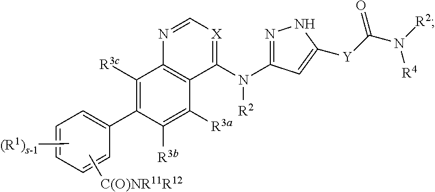
or a pharmaceutically acceptable salt thereof.

5. The compound according to claim 4 wherein the compound is represented by the following structural formula: 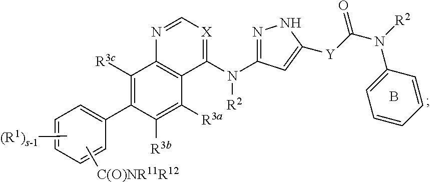
or a pharmaceutically acceptable salt thereof, wherein Phenyl Ring B is optionally substituted with one to three substituents.

6. The compound of claim 5 wherein R¹¹ and R¹², taken together with the nitrogen atom to which they are attached, form a 3 to 9 membered nitrogen-containing heterocycle, optionally containing 1 additional ring heteroatom selected from oxygen, nitrogen and sulfur, and optionally substituted with oxo, halo, (C₁-C₄)alkyl, (C₃-C₈)cycloalkyl, spiro (C₃-C₈)cycloalkyl, (C₃-C₈)cycloalkyl(C₁-C₄)alkyl, (C₁-C₄)hydroxyalkyl, —(CH₂)_{q}—OR, (C₁-C₄)alkoxyalkyl, (C₁-C₄)alkoxy, (C₃-C₈)cycloalkoxy, (C₃-C₈)cycloalkyl(C₁-C₄)alkoxy, (C₁-C₄)thioalkyl, (C₃-C₈)cycloalkylthio, (C₃-C₈)cycloalkyl(C₁-C₄)thioalkyl, halo(C₁-C₄)alkyl, halo(C₁-C₄)alkoxy, amino, (C₁-C₄)alkylamino, (C₃-C₈)cycloalkylamino, (C₃-C₈)cycloalkyl(C₁-C₄)alkylamino, [(C₃-C₈)cycloalkyl(C₁-C₄)alkyl][(C₁-C₄)alkyl]amino, [(C₃-C₈)cycloalkyl(C₁-C₄)alkyl][(C₃-C₈)cycloalkyl]amino, di(C₁-C₄)alkylamino, di(C₃-C₈)cycloalkylamino, di[(C₃-C₈)cycloalkyl(C₁-C₄)alkyl]amino, aminocarbonyl, (C₁-C₄)alkylaminocarbonyl, (C₃-C₈)cycloalkylaminocarbonyl, (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl, di[(C₃-C₈)cycloalkyl(C₁-C₄)alkyl]carbonyl, (C₁-C₄)alkylcarbonyl, (C₃-C₈)cycloalkylcarbonyl, (C₃-C₈)cycloalkyl(C₁-C₄)alkylcarbonyl, (C₁-C₄)alkoxycarbonyl, (C₃-C₈)cycloalkoxycarbonyl, (C₃-C₈)cycloalkyl(C₁-C₄)alkoxycarbonyl, S(O)ₚ(C₁-C₄)alkyl, NO₂, OH, CN or 3 to 9 membered nitrogen-containing heterocyclyl optionally containing 1 additional heteroatom selected from oxygen, nitrogen or sulfur.

7. The compound according to claim 5, wherein the compound is represented by the following structural formula: 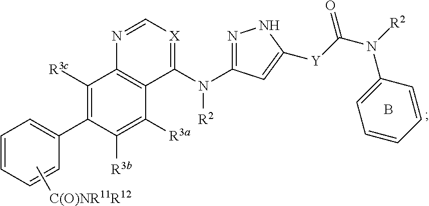
or a pharmaceutically acceptable salt thereof, wherein Ring B is optionally substituted with one to three substituents.

8. The compound according to claim 7, wherein the compound is represented by the following structural formula: 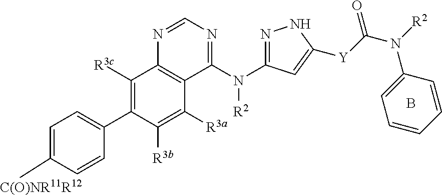
or a pharmaceutically acceptable salt thereof, wherein Ring B is optionally substituted with one to three substituents.

9. The compound according to claim 8, wherein the compound is represented by the following structural formula: 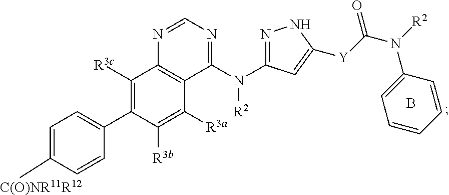
or a pharmaceutically acceptable salt thereof; wherein Ring B is optionally substituted with one to three substituents.

10. The compound according to claim 3 wherein the compound is represented by the following structural formula: 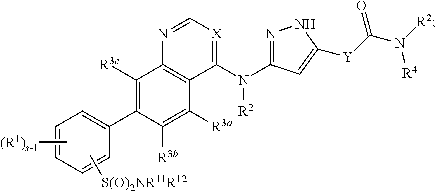
or a pharmaceutically acceptable salt thereof.

11. The compound according to claim 10, wherein the compound is represented by the following structural formula: 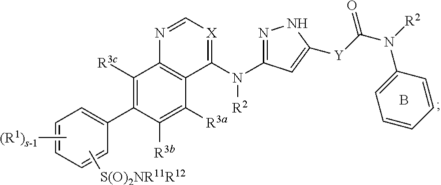
or a pharmaceutically acceptable salt thereof, wherein Phenyl Ring B is optionally substituted with one to three substituents.

12. The compound according to claim 11 wherein the compound is represented by the following structural formula: 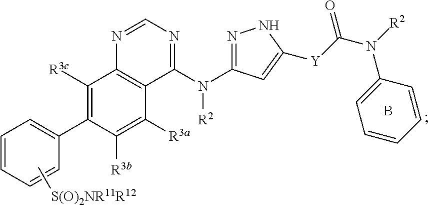
or a pharmaceutically acceptable salt thereof, wherein Ring B is optionally substituted with one to three substituents.

13. The compound according to claim 12, wherein the compound is represented by the following structural formula: 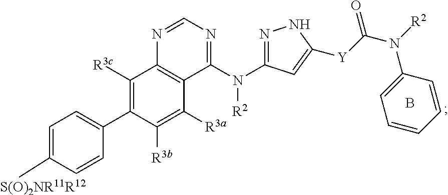
or a pharmaceutically acceptable salt thereof, wherein Ring B is optionally substituted with one to three substituents.

14. The compound according to claim 13, wherein the compound is represented by the following structural formula: 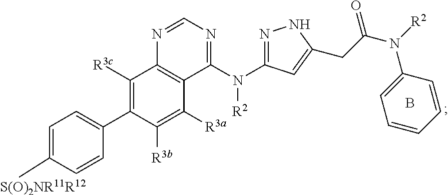
or a pharmaceutically acceptable salt thereof; wherein Ring B is optionally substituted with one to three substituents.

15. The compound according to claim 4, wherein Ring B is optionally substituted with up to three substituents selected from halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino, OH, NO₂ and CN.

16. The compound according to claim 4, wherein the 3 to 9 membered heterocycle formed from R¹¹ and R¹², taken together with the nitrogen atom to which they are attached is: i) optionally substituted at any substitutable ring carbon atoms with halogen, oxo, (C₁-C₄)alkyl, spiro (C₃-C₈)cycloalkyl, (C₃-C₈)cycloalkyl, hydroxy, (C₁-C₄)alkoxy or three to seven membered nitrogen-containing heterocylyl optionally containing one additional ringheteroatom selected from nitrogen and oxygen; and ii) optionally substituted at any substitutable ring nitrogen atom with (C₁-C₄)alkyl, (C₁-C₄)haloalkyl, (C₃-C₈)cycoalkyl, (C₃-C₈)cycoalkyl(C₁-C₄)alkyl, hydroxy(C₁-C₄)alkyl, —(CH₂)_{q}—OR, (C₁-C₄)alkoxyalkyl, (C₁-C₄)alkylcarbonyl, (C₃-C₈)cycloalkylcarbonyl, (C₃-C₈)cycloalkyl(c₁-C₄)alkylcarbonyl, (C₁-C₄)alkylaminocarbonyl, (C₃-C₈)cycloalkylaminocarbonyl (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl or di[(C₃-C₈)cycloalkyl(C₁-C₄)alkyl]carbonyl.

17. The compound of claim 1, wherein:
each R¹ is independently halo, OR¹¹, S(O)ₚR¹¹, CN, NO₂, CO₂R¹¹, CHO, (C₁-C₆)alkyl, halo(C₁-C₄)alkyl, (C₃-C₈)cycloalkyl, (3 to 9 membered)heterocyclyl, (C₆-C₁₀)aryl, (5 to 10 membered)heteroaryl, CONR¹¹R¹², OC(O)NR¹¹R¹², NR¹¹C(O)NR¹¹R¹², SO₂NR¹¹R¹², NR¹¹SO₂NR¹¹R¹², NR¹¹C(O)OR¹², NR¹¹C(S)OR¹², or NR¹¹SO₂R¹²;
R^{3a} is H, halo, OR¹³, S(O)ₚR¹³, CN, NO₂, CO₂R¹³, CHO, (C₁-C₆)alkyl, halo(C₁-C₄)alkyl, (C₃-C₈)cycloalkyl, CONR¹³R¹⁴, OC(O)NR¹³R¹⁴, NR¹³C(O)NR¹³R¹⁴, SO₂NR¹³R¹⁴, NR¹³SO₂NR¹³R¹⁴, NR¹³C(O)OR¹⁴, NR¹³C(S)OR¹⁴, or NR¹³SO₂R¹⁴;
the (C₃-C₈)cycloalkyl and the (3 to 9 membered)heterocyclyl represented by R¹ and R^{3a} are optionally and independently substituted with oxo, halo, (C₁-C₄)alkyl, (C₁-C₄)hydroxyalkyl, (C₁-C₄)alkoxyalkyl, (C₁-C₄)alkoxy, (C₁-C₄)thioalkyl, halo(C₁-C₄)alkyl, halo(C₁-C₄)alkoxy, amino, (C₁-C₃)alkylamino, di(C₁-C₃)alkylamino, aminocarbonyl, (C₁-C₃)alkylaminocarbonyl, di(C₁-C₃)alkylaminocarbonyl, (C₁-C₄)alkylcarbonyl, S(O)ₚ(C₁-C₄)alkyl, NO₂, OH, or CN

18. The compound of claim 1, wherein:
each R¹ is independently halo, (C₁-C₄)alkyl, (C₁-C₄)alkoxy, halo(C₁-C₄)alkyl, halo(C₁-C₄)alkoxy, CONR¹¹R¹², SO₂NR¹¹R¹², amino, (C₁-C₆)alkylamino, di(C₁-C₆)alkylamino OH, NO₂ or CN;
R^{3a} is H, halo, (C₁-C₄)alkyl, (C₁-C₄)alkoxy, halo(C₁-C₄)alkyl, halo(C₁-C₄)alkoxy, amino, (C₁-C₆)alkylamino, di(C₁-C₆)alkylamino OH, NO₂ or CN; and
R^{3b} and R^{3c} are each H.

19. The compound of claim 1, wherein R⁴ is (C₆-C₁₀)aryl, (5 to 10 membered)heteroaryl, (C₆-C₁₀)aryl(C₁-C₄)alkyl, (5 to 10 membered)heteroaryl(C₁-C₄)alkyl, (C₁-C₆)alkyl, (C₃-C₈)cycloalkyl, or (3 to 9 membered)heterocyclyl, each optionally and independently substituted with 1 to 4 substituents selected from the group consisting of halo, OR¹³, S(O)ₚR¹³, CN, NO₂, CO₂R¹³, CHO, (C₁-C₆)alkyl, halo(C₁-C₆)alkyl, (C₃-C₈)cycloalkyl, (5 to 9 membered)heterocyclyl, (C₆-C₁₀)aryl, (5 to 10 membered)heteroaryl, NR¹³R¹⁴, CONR¹³R¹⁴, OC(O)NR¹³R¹⁴, NR¹³C(O)NR¹³R¹⁴, CSNR¹³R¹⁴, OC(S)NR¹³R¹⁴, NR¹³C(S)NR¹³R¹⁴, SO₂NR¹³R¹⁴, NR¹³SO₂NR¹³R¹⁴, NR¹³C(O)R¹⁴, NR¹³C(S)R¹⁴, NR¹³C(O)OR¹⁴, NR¹³C(S)OR¹⁴, or NR¹³SO₂R¹⁴.

20. The compound of claim 16 wherein from R¹¹ and R¹², taken together with the nitrogen atom to which they are attached form an aziridinyl, azetidinyl, pyrrolidinyl, piperazinyl, piperidinyl, or morpholinyl group, each of which is: i) optionally substituted at any substitutable ring carbon atoms with halogen, oxo, (C₁-C₄)alkyl, spiro (C₃-C₈)cycloalkyl, (C₃-C₈)cycloalkyl, hydroxy, (C₁-C₄)alkoxy or three to seven membered nitrogen-containing heterocylyl selected from aziridinyl, azetidinyl, pyrrolidinyl, piperazinyl, piperidinyl, and morpholinyl; and ii) optionally substituted at any substitutable ring nitrogen atom with (C₁-C₄)alkyl, (C₁-C₄)haloalkyl, (C₃-C₈)cycoalkyl, (C₃-C₈)cycoalkyl(C₁-C₄)alkyl, hydroxy(C₁-C₄)alkyl, —(CH₂)_{q}—OR, (C₁-C₄)alkoxyalkyl, (C₁-C₄)alkylcarbonyl, (C₃-C₈)cycloalkylcarbonyl, (C₃-C₈)cycloalkyl(c₁-C₄)alkylcarbonyl, (C₁-C₄)alkylaminocarbonyl, (C₃-C₈)cycloalkylaminocarbonyl (C₃-C₈)cycloalkyl(C₁-C₄)alkylaminocarbonyl, [(C₃-C₈)cycloalkyl(C₁-C₄)alkyl][(C₁-C₄)alkyl]aminocarbonyl, [(C₃-C₈)cycloalkyl(C₁-C₄)alkyl][(C₃-C₈)cycloalkyl]aminocarbonyl, [(C₃-C₈)cycloalkyl][(C₁-C₄)alkyl]aminocarbonyl, di(C₁-C₄)alkylaminocarbonyl, di(C₃-C₈)cycloalkylaminocarbonyl or di[(C₃-C₈)cycloalkyl(C₁-C₄)alkyl]carbonyl.

21. A compound represented by a structural formula selected from: 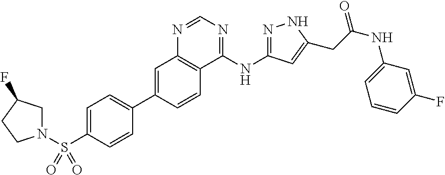
(R)-N-(3-fluorophenyl)-2-(3-(7-(4-(3-fluoropyrrolidin-1-ylsulfonyl)phenyl)quinazolin-4-ylamino)-1H-pyrazol-5-yl)acetamide; 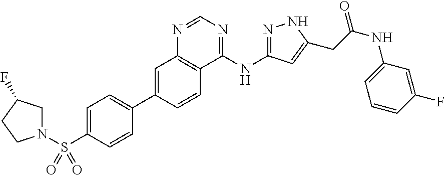
(S)-N-(3-fluorophenyl)-2-(3-(7-(4-(3-fluoropyrrolidin-1-ylsulfonyl)phenyl)quinazolin-4-ylamino)-1H-pyrazol-5-yl)acetamide; 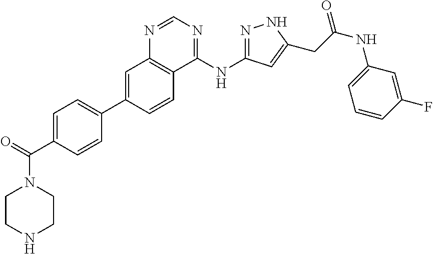
N-(3-fluorophenyl)-2-(3-(7-(4-(piperazine-1-carbonyl)phenyl)quinazolin-4-ylamino)-1H-pyrazol-5-yl)acetamide; 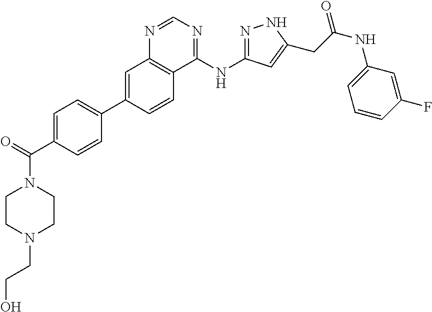
N-(3-fluorophenyl)-2-(3-(7-(4-(4-(2-hydroxyethyl)piperazine-1-carbonyl)phenyl)quinazolin-4-ylamino)-1H-pyrazol-5-yl)acetamide; 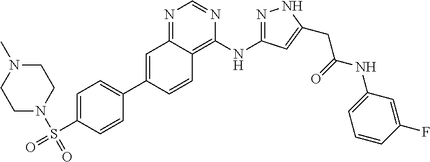
N-(3-fluorophenyl)-2-(3-(7-(4-(4-methylpiperazin-1-ylsulfonyl)phenyl)quinazolin-4-ylamino)-1H-pyrazol-5-yl)acetamide; 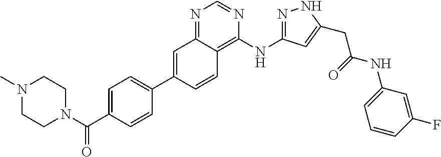
N-(3-fluorophenyl)-2-(3-(7-(4-(4-methylpiperazine-1-carbonyl)phenyl)quinazolin-4-ylamino)-1H-pyrazol-5-yl)acetamide; and 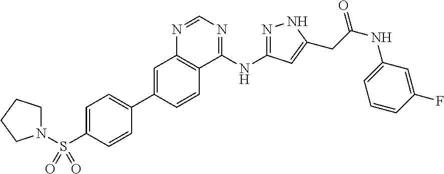
N-(3-fluorophenyl)-2-(3-(7-(4-(pyrrolidin-1-ylsulfonyl)phenyl)quinazolin-4-ylamino)-1H-pyrazol-5-yl)acetamide;
or a pharmaceutically acceptable salt thereof.

22. A compound represented by the following structural formula: 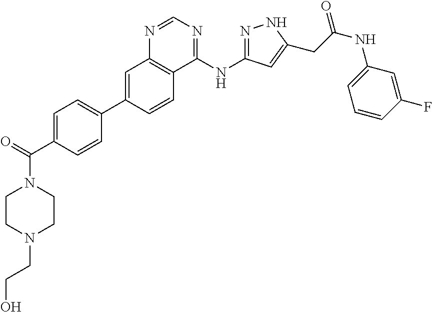
N-(3-fluorophenyl)-2-(3-(7-(4-(4-(2-hydroxyethyl)piperazine-1-carbonyl)phenyl)quinazolin-4-ylamino)-1H-pyrazol-5-yl)acetamide;
or a pharmaceutically acceptable salt thereof.

23. A pharmaceutical composition comprising a pharmaceutically acceptable carrier or diluent and the compound of claim 1.

24. A method of treating a subject with cancer, comprising administering to the subject an effective amount of the compound claim 1.

25. The method of claim 24, wherein the cancer is colorectal, breast or leukemia.
